(19) United States
(12) Patent Application Publication
(10) Pub. No.: US 2026/0034064 A1
(43) Pub. Date: Feb. 5, 2026

(54) COMPOSITIONS FOR THE TREATMENT OF CFTR-MEDIATED DISEASES

(71) Applicant: Vertex Pharmaceuticals Incorporated, Boston, MA (US)

(72) Inventors: Kirk DINEHART, Holliston, MA (US); Lauren Bailey TIPS, Framingham, MA (US); Catherine METZLER, Medford, MA (US); Katie MCCARTY, Watertown, MA (US)

(21) Appl. No.: 19/100,776

(22) PCT Filed: Aug. 4, 2023
(86) PCT No.: PCT/US2023/071721

§ 371 Date: Feb. 3, 2025

Related U.S. Application Data
(60) Provisional application No. 63/395,048, filed on Aug. 4, 2022.

(51) Int. Cl. A61K 9/20; A61K 9/16; A61K 31/404; A61K 31/4439; A61K 31/47

(52) U.S. Cl. CPC A61K 9/2018; A61K 9/1611; A61K 9/1623; A61K 9/1652; A61K 9/2009; A61K 9/2054; A61K 31/404; A61K 31/4439; A61K 31/47

(57) ABSTRACT

The present invention relates to pharmaceutical compositions containing N-(1,3-dimethylpyrazol-4-yl)sulfonyl-6-[3-(3,3, 3-trifluoro-2,2-dimethyl-propoxy)pyrazol-1-yl]-2-[(4S)-2,2,4-trimethylpyrrolidin-1-yl]pyridine-3-carboxamide, a solid dispersion of (R)-1-(2,2-difluorobenzo[d][1,3]dioxol-5-yl)-7V-(1-(2,3-dihydroxypropyl)-6-fluoro-2-(1-hydroxy-2-methylpropan-2-yl)-1H-en indol-5-yl)cyclopropanecarboxamide, and a solid dispersion of N-[2,4-Bis(1,1-dimethylethyl)-5-hydroxyphenyl]-1,4-dihydro-4-oxo-quinoline-3-carboxamide, including formulations of the solid dispersions into powders, granules and mini-tablets, methods for manufacturing and processing the powders, granules and mini-tablets, and methods for treating cystic fibrosis employing the pharmaceutical compositions.

This application claims the benefit of U.S. Provisional Application No. 63/395,048, filed on Aug. 4, 2022, the contents of which are incorporated by reference in its entirety.

The present invention relates to pharmaceutical compositions containing N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide, (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, and N-(5-hydroxy-2,4-di-tert-butyl-phenyl)-4-oxo-1H-quinoline-3-carboxamide, including formulations of the solid compositions into powders, granules, and mini-tablets, methods for manufacturing and processing the powders, granules, and mini-tablets, and methods for treating cystic fibrosis employing the pharmaceutical compositions.

One aspect of the invention provides pharmaceutical compositions comprising modulators of Cystic Fibrosis Transmembrane Conductance Regulator (CFTR). Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to enhanced mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 485 of these identified mutations, with sufficient evidence to define 401 mutations as disease-causing. The most prevalent disease-causing mutation is a deletion of phenylalanine at position 508 of the CFTR amino acid sequence, and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of approximately 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺-K⁺-ATPase pump and Cl-channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K'⁰ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

N-(5-hydroxy-2,4-di-tert-butyl-phenyl)-4-oxo-1H-quinoline-3-carboxamide (Compound III) is a potent and selective CFTR potentiator of wild-type and mutant forms of human CFTR and is useful in treating cystic fibrosis. N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound I) and (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II) are both potent and selective CFTR correctors for treating mutant forms of human CFTR-mediated diseases, such as cystic fibrosis.

Pediatric CF patients may require administration of pharmaceutical compositions in a dosage form that facilitates swallowing or that may be easily mixed with easily digested foods. The use of powders and crushed tablets in the administration of pharmaceutical compositions to children has often presented problems in administration and dosing. Administering crushed tablet formulations to children can lead to absorption problems, fragments that are either too difficult to swallow, or fail to solubilize and remain undigested resulting in therapeutic failure, or dosage inaccuracies. Additionally, the dosing of crushed tablets can lead to dosing inaccuracies because of difficulties associated with the handling of crushed tablets. The use of powder blends may also result in dosage inaccuracies. In other instances, active powder agents may remain adhered to the interior walls of a capsule, pouch, or packet at the time of administration, resulting in less than the required therapeutic dosage. Such dosing inaccuracies are particularly prevalent when the person administering the dose is inexperienced and when the dose is small, as in those used to treat pediatric patients. Dosage errors involving CF pharmaceutical active agents therefore become critical in pediatric populations, particularly considering that pharmaceutical CF active agents are administered in low doses (e.g., less than 100 mg or less than 50 mg per unit dose). These dosing inaccuracies become critical in pediatric patients having a low threshold for dose deviation.

Accordingly, there is a need for stable, bioavailable pharmaceutical compositions of Compound I, Compound II, and Compound III useful for treating patients, for example, CF patients having problems with swallowing adult tablets, including but not limited to pediatric patients, and methods for manufacturing and administering the same.

There is a need for stable, bioavailable pharmaceutical compositions of Compound I, Compound II, and Compound III useful for treating a particular population with an unmet medical need, such as children 12 to 24 months and 2 to 5 years of age, who cannot swallow adult tablets.

There is a need for stable, bioavailable pharmaceutical compositions of Compound I, Compound II, and Compound III that allow for accurate and flexible dosing in pediatric patients 12 to 24 months and 2 to 5 years of age by changing the number of mini-tablets in the unit dose, packet, pouch, or capsule.

### SUMMARY

The present invention relates to pharmaceutical compositions comprising Compound I, Compound II, and Compound III, and methods of manufacturing and administering pharmaceutical compositions comprising Compound I, Compound II, and Compound III. The pharmaceutical compositions comprising a Compound I, Compound II, and Compound III may also include one or more of the following excipients: one or more fillers, a sweetener, a disintegrant, a wetting agent, a glidant, and a lubricant.

The pharmaceutical compositions of the present invention can be formulated into tablets, mini-tablets, granules, pellets, troches and other dosage forms. Granulated particle forms of the pharmaceutical composition, such as mini-tablets, granules, sprinkles, pellets, beads, particles, particulates, troches and other dosage forms can be contained in capsules, pouches, packets, sachets, bottles or blister packs to provide a unit dosage form. Mini-tablets, granules, sprinkles, pellets, beads, particulates, or particles can also be compressed into other solid forms. In one embodiment, pharmaceutical composition can include granulated formulations described herein containing: crystalline Compound I Form A, a solid dispersion of Compound II, and a solid dispersion of Compound III and an excipient (for example, one or more fillers, a sweetener, a disintegrant, optionally a wetting agent, a glidant and a lubricant) and formulated into a capsule or a packet, the capsule or the packet containing a unit dosage form, i.e., a specified amount, of Compound I, ranging from at least 20 mg to at least 100 mg, amorphous Compound II, ranging from 10 mg to 50 mg, and amorphous Compound III, ranging from 15 mg to 75 mg. Mini-tablets, granules, sprinkles, pellets, beads, particulates, or particles and other dosage forms may comprise crystalline Compound I Form A, a solid dispersion of Compound II, and a solid dispersion of Compound III.

In one embodiment, the present invention provides a unit dosage form of a pharmaceutical composition formulated to deliver 20 mg, 80 mg, or 100 mg of crystalline Compound I Form A, a solid dispersion of Compound II, wherein the solid dispersion comprises 10 mg, 40 mg, or 50 mg of amorphous Compound II, and a solid dispersion of Compound III, wherein the solid dispersion comprises 15 mg, 60 mg, or 75 mg of amorphous Compound III. In another embodiment, the present invention provides a pharmaceutical composition formulated to deliver 100 mg of crystalline Compound I Form A, a solid dispersion of Compound II, wherein the solid dispersion comprises 50 mg of amorphous Compound II, and a solid dispersion of Compound III, wherein the solid dispersion comprises 75 mg of amorphous Compound III. In certain embodiments, the present invention provides a pharmaceutical composition formulated to deliver 80 mg of crystalline Compound I Form A, a solid dispersion of Compound II, wherein the solid dispersion comprises 40 mg of amorphous Compound II, and a solid dispersion of Compound III, wherein the solid dispersion comprises 60 mg of amorphous Compound III. In yet another embodiment, the pharmaceutical composition of the invention is formulated to deliver 20 mg of crystalline Compound I Form A, a solid dispersion of Compound II, wherein the solid dispersion comprises 10 mg of amorphous Compound II, and a solid dispersion of Compound III, wherein the solid dispersion comprises 15 mg of amorphous Compound III.

In some embodiments, the pharmaceutical compositions disclosed herein comprise a first solid dispersion comprising Compound II and a second solid dispersion comprising Compound III. In some embodiments one or both solid dispersions are spray-dried dispersions. In some embodiments, the solid dispersions and the spray dried dispersions of the disclosure can comprise excipients, such as polymers and/or surfactants.

In some embodiments, sodium lauryl sulfate (SLS) is used as a surfactant in the solid dispersion of Compound III. The amount of the surfactant (e.g., SLS) relative to the total weight of the solid dispersion may be between 0.1-15% w/w. For example, it is from 0.5% to 10%, such as from 0.5 to 5%, e.g., 0.5 to 4%, 0.5 to 3%, 0.5 to 2%, 0.5 to 1%, or 0.5%. In certain embodiments, the amount of the surfactant relative to the total weight of the solid dispersion is at least 0.1% or at least 0.5%. In some embodiments, the surfactant is SLS in an amount of 0.5% by weight of the solid dispersion of Compound III.

In some embodiments, the solid dispersions (e.g., spray dried dispersions) of the disclosure comprise a polymer(s). Any suitable polymers known in the art can be used in the disclosure. Exemplary suitable polymers include polymers selected from cellulose-based polymers, polyoxyethylene-based polymers, polyethylene-propylene glycol copolymers, vinyl-based polymers, PEO-polyvinyl caprolactam-based polymers, and polymethacrylate-based polymers. In some embodiments, the polymer(s) is selected from cellulosic polymers such as hydroxypropylmethylcellulose (HPMC) and/or hydroxypropylmethylcellulose acetate succinate (HPMCAS). In some embodiments, the solid dispersion comprising Compound II further comprises HPMC. In some embodiments, the solid dispersion comprising Compound III further comprises HPMCAS.

In some embodiments, Compound II (or Compound III) and polymer are present in roughly equal amounts in weight, for example, each of the polymer and the drug make up half of the percentage weight of the dispersion. For example, the polymer is present in 49.5 wt % and Compound II (or Compound III) is present in 50 wt %. In another embodiment Compound II (or Compound III) is present in an amount greater than half of the percentage weight of the dispersions. In some embodiments, a first solid dispersion comprises from 70 wt % to 90 wt % (e.g., from 75 wt % to 85 wt %) of Compound II. In some embodiments, a second solid dispersion comprises from 70 wt % to 90 wt % (e.g., from 75 wt % to 85 wt %) of Compound III. For example, in some embodiments, in a solid dispersion comprising Compound II, the polymer is present in 20 wt % and amorphous Compound II is present in 80 wt %. In other embodiments, a solid dispersion of Compound III comprises 19.5 wt % of polymer, 80 wt % of amorphous Compound III, and 0.5 wt % of SLS.

In one embodiment, the solid dispersion of Compound II comprises about 80 percent of amorphous Compound II by weight of the solid dispersion, and about 20 percent of IPMC by weight of the solid dispersion.

In one embodiment, the solid dispersion of Compound III comprises about 80 percent of amorphous Compound III by weight of the solid dispersion, and about 19.5 percent of HPMCAS by weight of the solid dispersion, and about 0.5 percent SLS by weight of the dispersion.

In some embodiments, each of the first and second solid dispersions independently comprise a plurality of particles having a mean particle diameter of 5 to 100 microns. In some embodiments, each of the first and second solid dispersions independently comprise a plurality of particles having a mean particle diameter of 15 to 40 microns. In some embodiments, each of the first and second solid dispersions independently comprise a plurality of particles having a mean particle diameter of 15 microns.

In still further embodiments, in addition to the active pharmaceutical agents (APIs), the pharmaceutical compositions disclosed herein also comprise one or more fillers (e.g., mannitol, celluloses, calcium carbonate, starches, sugars (e.g., dextrose, lactose, or the like)) in concentrations of at least about 10 wt % by weight of the composition; a sweetener (e.g., sucralose, sorbitol, saccharin, fructose, aspartame, or a combination thereof) in a concentration of about 10% or less by weight of this composition; a disintegrant (e.g., croscarmellose sodium, sodium starch glycolate, or a combination thereof) in concentrations of about 10 wt % or less by weight of the composition; optionally a wetting agent (e.g., sodium lauryl sulfate, SLS) in concentrations of about 10 wt % or less by weight of the composition; a glidant (e.g., colloidal silicon dioxide, talc, or a combination thereof) in concentrations of about 2 wt % or less by weight of the composition; and a lubricant (e.g., magnesium stearate, stearic acid, hydrogenated oil, sodium stearyl fumarate, or any combination thereof) in concentrations of about 5 wt % or less by weight of the composition.

Such pharmaceutical compositions can optionally comprise one or more colorants, fragrances, and/or flavors to enhance its visual appeal, taste, and scent.

In one aspect, the invention includes a pharmaceutical composition comprising crystalline Compound I Form A, a first solid dispersion of amorphous or substantially amorphous Compound II, a second solid dispersion of amorphous or substantially amorphous Compound III, one or more fillers, a sweetener, a disintegrant, a glidant and a lubricant, and optionally a wetting agent. In another aspect, the invention includes a pharmaceutical composition comprising crystalline Compound I Form A, a single solid dispersion comprising both amorphous or substantially amorphous Compound II and amorphous Compound III, one or more fillers, a sweetener, a disintegrant, a glidant and a lubricant, and optionally a wetting agent.

In some embodiments, the filler in the pharmaceutical compositions of the invention is selected from: mannitol, lactose, sucrose, dextrose, maltodextrin, sorbitol, xylitol, powdered cellulose, polyhydric alcohols, microcrystalline cellulose, silicified microcrystalline cellulose, cellulose acetate, methylcellulose, ethylcellulose, hydroxyethylcellulose, methylhydroxyethyl-cellulose, talc, starch, pregelatinized starch, dibasic calcium phosphate, calcium sulfate, calcium carbonate, or combinations thereof.

In one embodiment, the filler comprises microcrystalline cellulose which is present in an amount from about 40 to about 60 percent by weight of the composition.

In another embodiment, the filler comprises mannitol which is present in an amount from about 15 to about 40 percent by weight of the composition. In some embodiments, the filler comprises lactose in an amount from about 25 to about 45 percent by weight of the composition. In some embodiments, the filler is binary, comprising both mannitol and lactose.

In one embodiment, the sweetener comprises: glucose, sucrose, maltose, mannose, dextrose, fructose, lactose, trehalose, maltitol, lactitol, xylitol, sorbitol, mannitol, tagatose, glycerin, erythritol, isomalt, maltose, sucralose, aspartame, neotame, alitame, neohesperidin dihydrochalcone, cyclamate, thaumatin, acesulfame potassium, saccharin, saccharin sodium or combinations thereof. In another embodiment, the sweetener comprises sucralose which is present in an amount from about 0.1 to about 5 percent by weight of the composition.

In one embodiment, wherein the disintegrant comprises: croscarmellose sodium, sodium alginate, calcium alginate, alginic acid, starch, pregelatinized starch, sodium starch glycolate, polyvinylpyrrolidone, copolymers of polyvinylpyrrolidone, crospovidone, carboxymethylcellulose calcium, cellulose and its derivatives, carboxymethylcellulose sodium, soy polysaccharide, clays, gums, an ion exchange resin, an effervescent system based on food acids and an alkaline carbonate component, sodium bicarbonate, or combinations thereof. In a further embodiment, the disintegrant comprises croscarmellose sodium which is present in an amount from about 1.5 to about 8 percent by weight of the composition.

In one embodiment, wherein the optional wetting agent comprises: sodium lauryl sulfate, cetostearyl alcohol, cetomacrogol emulsifying wax, gelatin, casein, docusate sodium, benzalkonium chloride, calcium stearate, polyethylene glycols, phosphates, polyoxyethylene sorbitan fatty acid esters, gum acacia, cholesterol, tragacanth, polyoxyethylene 20 stearyl ethers, polyoxyethylene alkyl ethers, polyoxyethylene castor oil derivatives, pegylated hydrogenated castor oils, sorbitan esters of fatty acids, Vitamin E or tocopherol derivatives, vitamin E TPGS, tocopheryl esters, lecithin, phospholipids and their derivatives, poloxamers, stearic acid, oleic acid, oleic alcohol, cetyl alcohol, mono and diglycerides, propylene glycol esters of fatty acids, glycerol esters of fatty acids, ethylene glycol palmitostearate, polyoxylglycerides, propylene glycol monocaprylate, propylene glycol monolaurate, alkyl aryl polyether alcohols and polyglyceryl oleate or combinations thereof. In another embodiment, the wetting agent comprises sodium lauryl sulfate which is present in an amount of about 2 or less percent by weight of the composition.

In one embodiment, the glidant comprises: talc, colloidal silica (i.e., colloidal silicon dioxide), precipitated silica, magnesium oxide, magnesium silicate, leucine, and starch. In a further embodiment, the glidant comprises colloidal silica which is present in an amount from about 0.1 to about 5 percent by weight of the composition.

In one embodiment, the lubricant comprises: talc, fatty acid, stearic acid, magnesium stearate, calcium stearate, sodium stearate, stearic acid, glyceryl monostearate, sodium lauryl sulfate, sodium stearyl fumarate, hydrogenated oils, polyethylene glycol, fatty alcohol, fatty acid ester, glyceryl behenate, mineral oil, vegetable oil, leucine, sodium benzoate, or a combination thereof. In a further embodiment, the lubricant comprises magnesium stearate which is present in an amount from about 0.5 to about 1.5 percent by weight of the composition.

A set of experiments were performed to evaluate the effect of different excipients on composition performance and general processing. The croscarmellose sodium levels, magnesium stearate levels, colloidal silicon dioxide levels, and the lactose:mannitol ratio were evaluated.

In another aspect, the invention includes a pharmaceutical composition comprising:
crystalline Compound I Form A in an amount of about 10 to about 25 percent by weight of the pharmaceutical composition,
a solid dispersion of amorphous or substantially amorphous Compound II in an amount of about 5 to about 15 percent by weight of the pharmaceutical composition;
a solid dispersion of amorphous or substantially amorphous Compound III in an amount of about 10 to about 20 percent by weight of the pharmaceutical composition;
sucralose in an amount of about 1 to about 2 percent by weight of the pharmaceutical composition;
croscarmellose sodium in an amount from about 4 to about 8 percent of by weight of the pharmaceutical composition;
colloidal silicon dioxide in an amount of about 0.5 to about 1.5 percent by weight of the pharmaceutical composition;
magnesium stearate in an amount of about 0.5 to about 1.5 percent by weight of the pharmaceutical composition;
mannitol in an amount of about 10 to about 20 percent by weight of the pharmaceutical composition; and
lactose monohydrate in an amount of about 25 to about 45 percent by weight of the pharmaceutical composition.

In another aspect, the invention includes a pharmaceutical composition comprising:
crystalline Compound I Form A in an amount of about 14 to about 17 percent by weight of the pharmaceutical composition,
a solid dispersion of amorphous or substantially amorphous Compound II in an amount of about 8 to about 12 percent by weight of the pharmaceutical composition;
a solid dispersion of amorphous or substantially amorphous Compound III in an amount of about 12 to about 16 percent by weight of the pharmaceutical composition;
sucralose in an amount of about 1 to about 2 percent by weight of the pharmaceutical composition;
croscarmellose sodium in an amount of about 6 percent by weight of the pharmaceutical composition;
colloidal silicon dioxide in an amount of about 1.0 percent by weight of the pharmaceutical composition;
magnesium stearate in an amount of about 1.0 percent by weight of the pharmaceutical composition;
mannitol in an amount of about 11 to about 15 percent by weight of the pharmaceutical composition; and
lactose monohydrate in an amount of about 35 to about 40 percent by weight of the pharmaceutical composition.

The present invention contemplates dosage forms such as granules, pellets, mini-tablets, and other solid dose forms which overcome the problems described above with respect to dosing inaccuracies, in particular, for pediatric patients. These stable, solid unit dose forms can have any shape, including oval, spherical, cylindrical, elliptical, cubical, square, or rectangular among others. Tablets or mini-tablets may have flat, shallow, standard, deep convex, or double deep convex faces or combinations thereof.

In one aspect, the pharmaceutical composition can be formulated into a unit dosage form, for example, a capsule, a sachet, and the like, containing at least one or more mini-tablets to simplify the administration of the pharmaceutical composition. In some embodiments, the unit dosage form can include a capsule or a packet containing at least one mini-tablet, or a plurality of mini-tablets. In another embodiment, the unit dose can include a pouch, a packet or sachet containing a specific dose of crystalline Compound I Form A, a solid dispersion comprising amorphous Compound II, and a solid dispersion comprising Compound III, in granular form.

Such pharmaceutical compositions as described herein can be in the form of a mini-tablet, and/or a plurality of mini-tablets made up of any number of mini-tablets (e.g., at least 10, at least 20, at least 40, at least 60, at least 80, at least 90, at least 100, or any number greater than 100, inclusive of all of the ranges in between). Thus, pharmaceutical compositions as described herein can be in the form of an individual mini-tablet, or a plurality of mini-tablets.

In another aspect, the coated mini-tablets described herein are colored, such as by incorporating a colorant in the mini-tablet formulation or by coloring the surface of the mini-tablet.

In other embodiments, the pharmaceutical composition comprising a mini-tablet or plurality of mini-tablets can be in pouches, sachets, packets, bottles or blister packs, or optionally further compressed into different solid unit dose forms that can be easily administered to patients that have difficulty in swallowing adult sized tablet formulations. As such, these novel powder pharmaceutical compositions and unit dose forms containing said pharmaceutical compositions are organoleptically acceptable to said patients, are sprinkled into liquids or soft food and disintegrated or dispersed in those various liquids and soft foods or food compositions such as milk (including breast milk), baby formula or infant formula, apple sauce, water, plain yogurt, ice cream, baby food, ensuring that the entire prescribed dose has been disintegrated or dispersed and are capable of administration to patients having difficulty swallowing adult tablets. Baby food includes, but is not limited to, carrots or carrot puree. The pharmaceutical composition can also be administered in strawberry preserves, rice pudding, chocolate pudding and the like. In one embodiment, the unit dose form is sprinkled into soft food and administered. In another embodiment, the unit dose form is sprinkled into liquid and administered. In one embodiment, the unit dose form is sprinkled into soft food, mixed, and administered. In another embodiment, the unit dose form is sprinkled into liquid, mixed, and administered. Liquids may include, but are not limited to, baby formula, infant formula, milk or breast milk. In some instances, for smaller sized mini-tablets or granules, the contents of packets, pouches, capsules, bottles or sachets may be administered directly to the mouth followed by breast milk or formula. Methods of administration of the present invention can optionally also include, for smaller sized mini-tablets or granules, administering the contents of packets, pouches, capsules, bottles or sachets directly to the mouth followed by a liquid or beverage. In some embodiments, any methods of administration of the present invention can optionally include orally administering with fat-containing food such as a standard CF high-calorie, high-fat meal or snack. In other embodiments, any methods of administration of the present invention can optionally include orally administering concurrently with, before, or after fat-containing food such as a standard CF high-calorie, high-fat meal or snack. In one embodiment, the pharmaceutical compositions of the present invention and solid unit dose forms thereof find particular utility in the treatment of CFTR mediated disease in the pediatric patient population.

In one embodiment, the pharmaceutical composition is a unit dose form comprising one or a plurality of granules, pellets, particles, or mini-tablets, and wherein the unit dose form comprises from about 20 mg to about 100 mg of crystalline Compound I Form A. In some embodiments, the pharmaceutical composition is a unit dose form comprising one or a plurality of granules, pellets, particles or mini-tablets, and wherein the unit dose form comprises about 20 mg of crystalline Compound I Form A. In one embodiment, the pharmaceutical composition is a unit dose form comprising one or a plurality of granules, pellets, particles or mini-tablets, and wherein the unit dose form comprises about 80 mg of crystalline Compound I Form A. In some embodiments, the pharmaceutical composition is a unit dose form comprising one or a plurality of granules, pellets, particles, or mini-tablets, and wherein the unit dose form comprises about 100 mg of crystalline Compound I Form A.

In one embodiment, the pharmaceutical composition is a unit dose form comprising one or a plurality of granules, pellets, particles, or mini-tablets, and wherein the unit dose form comprises from about 10 mg to about 50 mg of amorphous or substantially amorphous Compound II. In some embodiments, the pharmaceutical composition is a unit dose form comprising one or a plurality of granules, pellets, particles, or mini-tablets, and wherein the unit dose form comprises about 10 mg of amorphous or substantially amorphous Compound II. In some embodiments, the pharmaceutical composition is a unit dose form comprising one or a plurality of granules, pellets, particles, or mini-tablets, and wherein the unit dose form comprises about 40 mg of amorphous or substantially amorphous Compound II. In some embodiments, the pharmaceutical composition is a unit dose form comprising one or a plurality of granules, pellets, particles or mini-tablets, and wherein the unit dose form comprises about 50 mg of amorphous or substantially amorphous Compound II.

When formulating a combination product that has multiple API's in a plurality of granules, pellets, particles, or mini-tablets, there is the option to package said plurality as mixtures of granules, pellets, particles, or mini-tablets of a single API (i.e. granules of API I, granules of API II, and granules of API III), which may be more straightforward to formulate. The pharmaceutical composition disclosed herein contains a combination of Compound I, Compound II, and Compound III within each granule, pellet, particle, or mini-tablet. When formulating such a combination composition, there may be differences in performance and bioavailability than from single API composition granule, pellet, particle, or mini-tablets that are formulated separately and mixed together. Thus, experimentation is required to evaluate the suitability of a combination composition.

In one embodiment, the pharmaceutical composition is a unit dose form comprising one or a plurality of granules, pellets, particles, or mini-tablets, and wherein the unit dose form comprises from about 15 mg to about 75 mg of amorphous or substantially amorphous Compound III. In some embodiments, the pharmaceutical composition is a unit dose form comprising one or a plurality of granules, pellets, particles, or mini-tablets, and wherein the unit dose form comprises about 15 mg of amorphous or substantially amorphous Compound III. In some embodiments, the pharmaceutical composition is a unit dose form comprising one or a plurality of granules, pellets, particles, or mini-tablets, and wherein the unit dose form comprises about 60 mg of amorphous or substantially amorphous Compound III. In some embodiments, the pharmaceutical composition is a unit dose form comprising one or a plurality of granules, pellets, particles or mini-tablets, and wherein the unit dose form comprises about 75 mg of amorphous or substantially amorphous Compound III.

In another embodiment, the solid dispersion(s) comprising Compound II and Compound III are present in the pharmaceutical composition of the invention in a combined amount of about 20 to about 35 percent by weight of the pharmaceutical composition and the unit dose form is a mini-tablet having a shape that is cylinder-like, oval-like, cone-like, sphere-like, ellipsis-like, polygon-like, or combinations thereof, wherein the mini-tablet has as its longest dimension or diameter a length of from approximately 1.5 mm to approximately 4.0 mm.

In a further embodiment, the combined solid dispersions are present in an amount of about 20 to about 35 percent by weight of the pharmaceutical composition and the unit dose form is a mini-tablet having a shape that is cylinder-like, oval-like, cone-like, sphere-like, ellipsis-like, polygon-like or combinations thereof, wherein the mini-tablet has as its longest dimension or diameter a length of approximately 2.0 mm.

In some embodiments, individual granules or mini-tablets are about 5.0 mg to about 10 mg in weight. In some embodiments, individual granules or mini-tablets are about 6.0 mg to about 9.0 mg in weight. In some embodiments, individual granules or mini-tablets are about 6.5 mg to about 8.0 mg in weight. In some embodiments, each granule is about 7.0 mg in weight. In some embodiments, each granule is approximately 2.0 mm in diameter and about 7.0 mg in weight.

Another aspect of the present invention provides a method of administering a pharmaceutical composition by orally administering to a patient, for example, a human pediatric patient, at least once per day, a unit dose (via capsule, sachet, blister pack, pouch, packet, bottle, or other container) comprising powder form of the pharmaceutical composition and/or a mini-tablet or plurality of mini-tablets, comprising crystalline Compound I Form A, a solid dispersion comprising amorphous Compound II, a solid dispersion comprising amorphous Compound III, one or more fillers, a sweetener, a disintegrant, optionally a wetting agent, a glidant, and a lubricant, wherein the unit dose comprises about 100 mg of crystalline Compound I Form A, about 50 mg of substantially amorphous or amorphous Compound II, and about 75 mg of amorphous or substantially amorphous Compound III.

Another aspect of the present invention provides a method of administering a pharmaceutical composition by orally administering to a patient, for example, a human pediatric patient, at least twice per day, a unit dose (via capsule, sachet, blister pack, pouch, packet, bottle, or other container) comprising powder form of the pharmaceutical composition and/or a mini-tablet or plurality of mini-tablets, comprising crystalline Compound I Form A, a solid dispersion comprising substantially amorphous or amorphous Compound II, a solid dispersion comprising substantially amorphous or amorphous Compound III, one or more fillers, a sweetener, a disintegrant, optionally a wetting agent, a glidant, and a lubricant, wherein the unit dose comprises about 80 mg of crystalline Compound I Form A, about 40 mg of substantially amorphous or amorphous Compound II, and about 60 mg of amorphous or substantially amorphous Compound III.

Another aspect of the present invention provides a method of administering a pharmaceutical composition by orally administering to a patient, for example, a human pediatric patient, at least twice per day, a unit dose (via capsule, sachet, blister pack, pouch, packet, bottle, or other container) comprising powder form of the pharmaceutical composition and/or a mini-tablet or plurality of mini-tablets, comprising crystalline Compound I Form A, a solid dispersion comprising substantially amorphous or amorphous Compound II, a solid dispersion comprising substantially amorphous or amorphous Compound III, one or more fillers, a sweetener, a disintegrant, optionally a wetting agent, a glidant, and a lubricant, wherein the unit dose comprises about 20 mg of crystalline Compound I Form A, about 10 mg of substantially amorphous or amorphous Compound II, and about 15 mg of amorphous or substantially amorphous Compound III.

### DETAILED DESCRIPTION

The present invention relates to pharmaceutical compositions containing N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound I), (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II), and N-(5-hydroxy-2,4-di-tert-butyl-phenyl)-4-oxo-1H-quinoline-3-carboxamide (Compound III), including formulations of the solid compositions into powders, granules and mini-tablets, methods for manufacturing and processing the powders, granules, and mini-tablets, and methods for treating cystic fibrosis employing the pharmaceutical composition. In some embodiments, Compound I is crystalline Compound I Form A. In some embodiments, Compound II is amorphous or substantially amorphous Compound II in a solid dispersion. In some embodiments, Compound III is amorphous or substantially amorphous Compound II in a solid dispersion.

### I. Definitions

As used herein, the term “active pharmaceutical ingredient” or “API” refers to a biologically active compound. APIs employed in the pharmaceutical compositions of the invention include a CF potentiator, N-[2,4-bis(1,1-dimethylethyl)-5-hydroxyphenyl]-1,4-dihydro-4-oxoquinoline-3-carboxamide (Compound III), and two CF corrector compounds, N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound I) and (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II).

The terms “about” and “approximately”, when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. The terms “about” and “approximately” may refer to an acceptable error for a particular value as determined by one of skill in the art, which depends in part on how the values is measured or determined. In some embodiments, the terms “about” and “approximately” mean within 20%, 15%, 10%, 5%, 4%, 3%, 2%, 1%, or 0.5% of a given value or range. As used herein, the symbol “˜” appearing immediately before a numerical value has the same meaning as the terms “about” and “approximately.”

As used herein, the term “amorphous” refers to a solid material having no long-range order in the position of its molecules. Amorphous solids are generally supercooled liquids in which the molecules are arranged in a random manner so that there is no well-defined arrangement, e.g., molecular packing, and no long-range order. Amorphous solids are generally isotropic, i.e., exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See, US 2004/0006237 for a comparison of XRPDs of an amorphous material and crystalline material.

As used herein, the term “substantially amorphous” refers to a solid material having little or no long-range order in the position of its molecules. For example, substantially amorphous materials have less than about 15% crystallinity (e.g., less than about 10% crystallinity or less than about 5% crystallinity). It is also noted that the term ‘substantially amorphous’ includes the descriptor, ‘amorphous,’ which refers to materials having no (0%) crystallinity.

### Compound I

As used herein, the term “Compound I” is used interchangeably with N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide, which has the following structure:

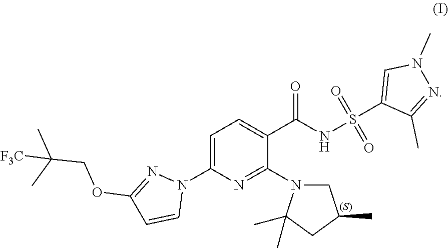

Compound I is described in International Patent Publications WO 2018/107100 and WO 2019/018395 incorporated herein by reference. WO 2018/107100 and WO 2019/018395 also describe methods of making Compound I, methods of making crystalline Form A of Compound I, and demonstrate that Compound I is a CFTR corrector therapeutic.

In some embodiments, the pharmaceutical compositions of the invention comprise crystalline Compound I Form A. Crystalline Compound I Form A is characterized by an X-ray powder diffractogram having a signal at least one two-theta value chosen from 6.6±0.2, 7.6±0.2, 9.6±0.2, 12.4±0.2, 13.1±0.2, 15.2±0.2, 16.4±0.2, 18.2±0.2, and 18.6±0.2. In some embodiments, crystalline Compound I Form A is characterized by an X-ray powder diffractogram having a signal at at least three two-theta values chosen from 6.6±0.2, 7.6±0.2, 9.6±0.2, 12.4±0.2, 13.1±0.2, 15.2±0.2, 16.4±0.2, 18.2±0.2, and 18.6±0.2. In some embodiments, crystalline Compound I Form A is characterized by an X-ray powder diffractogram having a signal at at least three two-theta values chosen from 6.6±0.2, 9.6±0.2, 13.1±0.2, 15.2±0.2, 18.2±0.2, and 18.6±0.2. In some embodiments, crystalline Compound I Form A is characterized by an X-ray powder diffractogram having a signal at three two-theta values of 6.6±0.2, 13.1±0.2, 18.2±0.2. In some embodiments, crystalline Compound I Form A is characterized by an X-ray powder diffractogram having a signal at six two-theta values of 6.6±0.2, 9.6±0.2, 13.1±0.2, 15.2±0.2, 18.2±0.2, and 18.6±0.2. Crystalline Compound I Form A was found to be the most thermodynamically stable form and to provide good bioavailability.

### Compound II

As used herein, the term “Compound II” is used interchangeably with (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide. Compound II can be depicted as having the following structure:

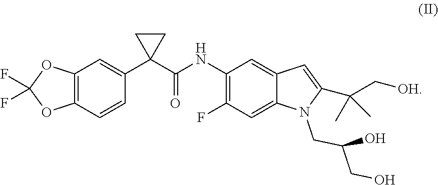 [SMILES: CC(C)(CO)c1cc2cc(NC(=O)C3(c4ccc5c(c4)OC(F)(F)O5)CC3)c(F)cc2n1C[C@@H](O)CO]

Compound II has been previously described in United States Patent Publication US 2009/0131492, and International Patent Publications WO 2011/119984 and WO 2015/160787, all of which are incorporated herein by reference.

### Compound III

As used herein, the term “Compound III” is used interchangeably with N-(5-hydroxy-2,4-di-tert-butyl-phenyl)-4-oxo-1H-quinoline-3-carboxamide and N-[2,4-Bis(1,1-dimethylethyl)-5-hydroxyphenyl]-1,4-dihydro-4-oxoquinoline-3-carboxamide. Compound III has the following structure:

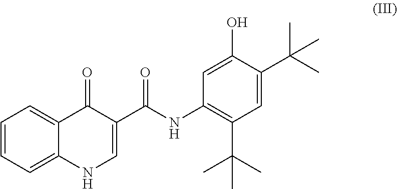 [SMILES: CC(C)(C)c1cc(C(C)(C)C)c(NC(=O)c2c[nH]c3ccccc3c2=O)cc1O]

has been previously described in International Patent Publication WO 2006/002421, WO 2007/079139, and WO 2013/130669, all of which are incorporated herein by reference.

As used herein, the term “dispersion” refers to a disperse system in which one substance, the dispersed phase, is distributed, in discrete units, throughout a second substance (the continuous phase or vehicle). The size of the dispersed phase can vary considerably (e.g., single molecules, colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include: an amorphous drug in an amorphous polymer; an amorphous drug in crystalline polymer; a crystalline drug in an amorphous polymer; or a crystalline drug in crystalline polymer. In this invention, a solid dispersion can include an amorphous drug in an amorphous polymer or an amorphous drug in crystalline polymer. In some embodiments, a solid dispersion includes the polymer constituting the dispersed phase, and the drug constitutes the continuous phase. Or, a solid dispersion includes the drug constituting the dispersed phase, and the polymer constitutes the continuous phase.

As used herein, the term “solid dispersion” generally refers to a solid dispersion of two or more components. In some embodiments a solid dispersion comprises a single API, (e.g., Compound II or Compound III). In some embodiments, the solid dispersion comprises two API (e.g., Compound II and Compound III). In some embodiments, the solid dispersion contains a polymer, but possibly containing other components such as surfactants or other pharmaceutical excipients, where the drug(s) (e.g., Compound II and/or Compound III) is substantially amorphous (e.g., having about 15% or less (e.g., about 10% or less, or about 5% or less)) of crystalline drug or amorphous (i.e., having no crystalline drug), and the physical stability and/or dissolution and/or solubility of the substantially amorphous or amorphous drug is enhanced by the other components. Solid dispersions typically include a compound dispersed in an appropriate carrier medium, such as a solid-state carrier. For example, a carrier comprises a polymer (e.g., a water-soluble polymer or a partially water-soluble polymer) and can include optional excipients such as functional excipients (e.g., one or more surfactants) or nonfunctional excipients (e.g., one or more fillers). Another exemplary solid dispersion is a co-precipitate or a co-melt of Compound II and/or Compound III, optionally comprising at least one polymer.

A “Co-precipitate” is a product after dissolving an API and a polymer in a solvent or solvent mixture followed by the removal of the solvent or solvent mixture. Sometimes the polymer can be suspended in the solvent or solvent mixture. The solvent or solvent mixture includes organic solvents and supercritical fluids. A “co-melt” is a product after heating a drug and a polymer to melt, optionally in the presence of a solvent or solvent mixture, followed by mixing, removal of at least a portion of the solvent if applicable, and cooling to room temperature at a selected rate.

As used herein, “crystallinity” refers to the degree of structural order in a solid. For example, Compound II and Compound III, which are substantially amorphous, have less than about 15% crystallinity, or its solid-state structure is less than about 15% crystalline. In another example, Compound II and/or Compound III are fully amorphous, i.e., have zero (0%) crystallinity.

As used herein, a “CF potentiator” refers to a compound that exhibits biological activity characterized by increasing gating functionality of the mutant CFTR protein present in the cell surface to approximately wild-type levels (i.e., a compound that augments or induces the channel activity of CFTR protein located at the cell surface, resulting in increased functional activity).

As used herein, the term “CFTR corrector” refers to a compound that augments or induces the amount of functional CFTR protein to the cell surface, resulting in increased functional activity.

As used herein, a “solid dose form” includes capsules, packets, sachets, and pouches containing the pharmaceutical composition either in powder form or in a compressed form, such as granules, pellets, particles, mini-tablets and the like, the solid dose form containing a specified amount of Compound I, Compound II, and Compound III.

As used herein, an “excipient” is an inactive ingredient in a pharmaceutical composition. Examples of excipients include a filler, a sweetener, a disintegrant, a glidant, a lubricant, and the like.

As used herein, a “diluent” or “filler” is an excipient that adds bulkiness to a pharmaceutical composition. Examples of fillers include mannitol, lactose, celluloses, such as microcrystalline cellulose and ethyl cellulose, cellulose acetate, calcium carbonate, potato starch, sorbitol, polyhydric alcohols, dextrose, or combinations thereof.

As used herein, a “disintegrant” is an excipient that hydrates a pharmaceutical composition and aids in tablet dispersion. Examples of disintegrants include sodium croscarmellose and/or sodium starch glycolate.

As used herein, a “sweetener” is an excipient that imparts a pharmaceutical composition with a sweet taste and/or masks other unpleasant tastes. Examples of sweeteners include sucralose, sorbitol, xylitol, and combinations thereof.

As used herein, a “glidant” is an excipient that imparts a pharmaceutical composition with enhanced flow properties. Examples of glidants include colloidal silica, precipitated silica and/or talc.

As used herein, a “colorant” is an excipient that imparts a pharmaceutical composition with a desired color. Examples of colorants include commercially available pigments such as FD&C Blue #1 Aluminum Lake, FD&C Blue #2, other FD&C Blue colors, titanium dioxide, iron oxide, and/or combinations thereof.

As used herein, a “lubricant” is an excipient that is added to pharmaceutical compositions to minimize adherence to surfaces, especially for pharmaceutical compositions that are pressed into tablets. The lubricant aids in ejection of a tablet of a pharmaceutical composition from a compression die. Examples of lubricants include magnesium stearate, stearic acid (stearin), hydrogenated oil, sodium stearyl fumarate, or any combination thereof.

As used herein, “mean particle diameter” is the average particle diameter as measured using techniques such as laser light scattering, image analysis, or sieve analysis.

As used herein, “bulk density” is the mass of particles of material divided by the total volume the particles occupy. The total volume includes particle volume, inter-particle void volume and internal pore volume. Bulk density is not an intrinsic property of a material; it can change depending on how the material is processed.

As used herein, a “wetting agent” is an excipient that imparts pharmaceutical compositions with enhanced solubility and/or wetability. Examples of wetting agents include sodium lauryl sulfate (SLS), sodium stearyl fumarate (SSF), polyoxyethylene 20 sorbitan mono-oleate (e.g., Tween™), or any combination thereof.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof.

As used herein, the term “mini-tablet” is equivalent to the term “granule.”

As use herein, “unit dose form” and “unit dosage form” are used interchangeably to refer to a package of mini-tablets or granules required to make up a specific dosage of API, such as, e.g., the one daily, or twice daily dosage of API.

As used herein, “CFTR” or “CFTR protein” stands for cystic fibrosis transmembrane conductance regulator protein.

Pharmaceutically acceptable salts are well known in the art. For example, S. M. Berge, et al. describes pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, edisylate (ethanedisulfonate), ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4alkyl)4 salts. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### II. Solid Dispersions

In some embodiments, the pharmaceutical compositions disclosed herein comprise a first solid dispersion comprising substantially amorphous or amorphous Compound II and a second solid dispersion comprising substantially amorphous or amorphous Compound III. In some embodiments one or both solid dispersions are spray-dried dispersions.

In some embodiments, each of the first and second solid dispersions independently comprise a plurality of particles having a mean particle diameter of 5 to 100 microns. In some embodiments, each of the first and second solid dispersions independently comprise a plurality of particles having a mean particle diameter of 15 to 40 microns. In some embodiments, each of the first and second solid dispersions independently comprise a plurality of particles having a mean particle diameter of 15 microns.

In some embodiments, the solid dispersions and the spray dried dispersions of the disclosure can comprise other excipients, such as polymers and/or surfactants. Any suitable polymers and surfactants known in the art can be used. Certain exemplary polymers and surfactants are as described below.

Solid dispersions of substantially amorphous or amorphous Compounds II, and/or substantially amorphous or amorphous Compound III, may be prepared by any suitable method known in the art, e.g., spray drying, lyophilizing, hot melting, or cyrogrounding/cryomilling techniques. Typically, such spray drying, lyophilizing, hot melting or cyrogrounding/cryomilling techniques results in an amorphous form of API (e.g., Compounds II and III).

Spray drying is a process that converts a liquid feed to a dried particulate form. Optionally, a secondary drying process such as fluidized bed drying or vacuum drying may be used to reduce residual solvents to pharmaceutically acceptable levels. Typically, spray drying involves contacting a highly dispersed liquid suspension or solution, and a sufficient volume of hot gas to produce evaporation and drying of the liquid droplets. The preparation to be spray dried can be any solution, coarse suspension, slurry, colloidal dispersion, or paste that may be atomized using the selected spray drying apparatus. In one procedure, the preparation is sprayed into a current of warm filtered gas that evaporates the solvent and conveys the dried product to a collector (e.g., a cyclone). The spent gas is then exhausted with the solvent, or alternatively the spent air is sent to a condenser to capture and potentially recycle the solvent. Commercially available types of apparatus may be used to conduct the spray drying. For example, commercial spray dryers are manufactured by Buchi Ltd. And Niro (e.g., the PSD line of spray driers manufactured by Niro) (see, US 2004/0105820; US 2003/0144257).

Techniques and methods for spray drying may be found in Perry's Chemical Engineering Handbook, 6th Ed., R. H. Perry, D. W. Green & J. O. Maloney, eds.), McGraw-Hill book co. (1984); and Marshall “Atomization and Spray-Drying” 50, Chem. Eng. Prog. Monogr. Series 2 (1954).

Removal of the solvent may require a subsequent drying step, such as tray drying, fluid bed drying, vacuum drying, microwave drying, rotary drum drying or biconical vacuum drying. In one embodiment, the solid dispersions and the spray dried dispersions of the disclosure are fluid bed dried. In one process, the solvent includes a volatile solvent, for example a solvent having a boiling point of less than 100° C. In some embodiments, the solvent includes a mixture of solvents, for example a mixture of volatile solvents or a mixture of volatile and non-volatile solvents. Where mixtures of solvents are used, the mixture can include one or more non-volatile solvents, for example, where the non-volatile solvent is present in the mixture at less than 15%, e.g., less than 12%, less than 10%, less than 8%, less than 5%, less than 3%, or less than 2%.

The particle size and the temperature drying range may be modified to prepare an optimal solid dispersion. As would be appreciated by skilled practitioners, a small particle size would lead to improved solvent removal. It has been found, however, that smaller particles may result in low bulk density that, under some circumstances, do not provide optimal solid dispersions for downstream processing.

A solid dispersion (e.g., a spray dried dispersion) disclosed herein may optionally include a surfactant. A surfactant or surfactant mixture would generally decrease the interfacial tension between the solid dispersion and an aqueous medium. An appropriate surfactant or surfactant mixture may also enhance aqueous solubility and bioavailability of the API(s) (e.g., substantially amorphous or amorphous Compound II and/or substantially amorphous or amorphous Compound III) from a solid dispersion. The surfactants for use in connection with the disclosure include, but are not limited to, sorbitan fatty acid esters (e.g., Spans®), polyoxyethylene sorbitan fatty acid esters (e.g., Tweens®), sodium lauryl sulfate (SLS), sodium dodecylbenzene sulfonate (SDBS) dioctyl sodium sulfosuccinate (Docusate sodium), dioxycholic acid sodium salt (DOSS), Sorbitan Monostearate, Sorbitan Tristearate, hexadecyltrimethyl ammonium bromide (HTAB), Sodium N-lauroylsarcosine, Sodium Oleate, Sodium Myristate, Sodium Stearate, Sodium Palmitate, Gelucire 44/14, ethylenediamine tetraacetic acid (EDTA), Vitamin E d-alpha tocopheryl polyethylene glycol 1000 succinate (TPGS), Lecithin, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, Pluronic F68, Pluronic F108, and Pluronic F127 (or any other polyoxyethylene-polyoxypropylene co-polymers (Pluronics®) or saturated polyglycolized glycerides (Gelucirs®)). Specific examples of such surfactants that may be used in connection with this disclosure include, but are not limited to, Span 65, Span 25, Tween 20, Capryol 90, Pluronic F108, sodium lauryl sulfate (SLS), Vitamin E TPGS, pluronics and copolymers.

In some embodiments, SLS is used as a surfactant in the solid dispersion of substantially amorphous or amorphous Compound III. The amount of the surfactant (e.g., SLS) relative to the total weight of the solid dispersion may be between 0.1-15% w/w. For example, it is from 0.5% to 10%, such as from 0.5 to 5%, e.g., 0.5 to 4%, 0.5 to 3%, 0.5 to 2%, 0.5 to 1%, or 0.5%. In certain embodiments, the amount of the surfactant relative to the total weight of the solid dispersion is at least 0.1% or at least 0.5%. In some embodiments, the surfactant is SLS in an amount of 0.5% by weight.

Another aspect of the disclosure provides a single spray dried dispersion comprising substantially amorphous or amorphous Compound II and substantially amorphous or amorphous Compound III, wherein the dispersion is prepared without a polymer, and wherein the spray dried dispersion is generated by (i) providing a mixture that consists essentially of substantially amorphous or amorphous Compound II and substantially amorphous or amorphous Compound III and a solvent; and (ii) forcing the mixture through a nozzle under spray drying conditions to generate the spray dried dispersion.

In some embodiments, solid dispersions for inclusion in a pharmaceutically acceptable composition of the disclosure may be prepared by non-spray drying techniques, such as, for example, cyrogrounding/cryomilling techniques. A solid dispersion comprising substantially amorphous Compound II or substantially amorphous or amorphous Compound III may also be prepared by hot melt extrusion techniques.

In some embodiments, the solid dispersions (e.g., spray dried dispersions) of the disclosure comprise a polymer(s). Any suitable polymers known in the art can be used in the disclosure. Exemplary suitable polymers include polymers selected from cellulose-based polymers, polyoxyethylene-based polymers, polyethylene-propylene glycol copolymers, vinyl-based polymers, PEO-polyvinyl caprolactam-based polymers, and polymethacrylate-based polymers.

The cellulose-based polymers include a methylcellulose, a hydroxypropyl methylcellulose (HPMC) (hypromellose), a hypromellose phthalate (HPMC-P), a hypromellose acetate succinate, and co-polymers thereof. The polyoxyethylene-based polymers include a polyethylene-propylene glycol, a polyethylene glycol, a poloxamer, and co-polymers thereof. The vinyl-based polymers include a polyvinylpyrrolidine (PVP), and PVP/VA. The PEO-polyvinyl caprolactam-based polymers include a polyethylene glycol, polyvinyl acetate and polyvinylcaprolactame-based graft copolymer (e.g., Soluplus®). The polymethacrylate-based polymers are synthetic cationic and anionic polymers of dimethylaminoethyl methacrylates, methacrylic acid, and methacrylic acid esters in varying ratios. Several types are commercially available and may be obtained as the dry powder, aqueous dispersion, or organic solution. Examples of such polymethacrylate-based polymers include a poly(methacrylic acid, ethyl acrylate) (1:1), a dimethylaminoethyl methacrylate-methylmethacrylate copolymer, and an Eudragit®.

In some embodiments, the cellulose-based polymer is a hypromellose acetate succinate (also known as hydroxypropyl methylcellulose acetate succinate or HMPCAS) and a hypromellose (also known as hydroxypropyl methylcellulose or HPMC), or a combination of hypromellose acetate succinate and a hypromellose. HPMCAS is available in various grades based on the content of acetyl and succinoyl groups (wt %) in the HPMCAS molecule and on particle size. For example, HPMCAS grades L, M, and H are available. HPMCAS-H is a grade that contains about 10-14 wt % of acetyl groups and about 4-8 wt % of succinoyl groups. Each HPMCAS grade is available in two particle sizes, F (fine) and G (granular). HPMC comes in various types (for example, HPMC E, F, J, and K-types). HPMC E type means that there are about 28-30% methoxy groups and about 7-12% hydroxpropoxy groups. There are various E grades ranging from low to high viscosity. For example, E3 means the viscosity is about 2.4-3.6 millipascal seconds (mPa s) for HPMC measured at 2% in water at 20° C.; E15 means the viscosity is about 12-18 mPa s for the HPMC measured at 2% in water at 20° C.; and E50 means the viscosity is about 40-60 mPa s for the HPMC measured at 2% in water at 20° C.

In some embodiments, the cellulose-based polymer is a hypromellose acetate succinate and a hypromellose, or a combination of hypromellose acetate succinate and a hypromellose. In some embodiments, the cellulose-based polymer is hypromellose E15, hypromellose acetate succinate L or hypromellose acetate succinate H. In some embodiments, the polyoxyethylene-based polymer or polyethylene-propylene glycol copolymer is a polyethylene glycol or a pluronic. In some embodiments, the polyoxyethylene-based polymer or polyethylene-propylene glycol copolymer is polyethylene glycol 3350 or poloxamer 407. In some embodiments, the vinyl-based polymer is a vinylpolyvinylpyrrolidine-based polymer, such as polyvinylpyrrolidine K30 or polyvinylpyrrolidine VA 64. In some embodiments, the polymethacrylate polymer is Eudragit L100-55 or Eudragit® E PO. In some embodiments, the polymer(s) is selected from cellulosic polymers such as HPMC and/or HPMCAS.

In one embodiment, a polymer is able to dissolve in aqueous media. The solubility of the polymers may be pH independent or pH dependent. The latter include one or more enteric polymers. The term “enteric polymer” refers to a polymer that is preferentially soluble in the less acidic environment of the intestine relative to the more acid environment of the stomach, for example, a polymer that is insoluble in acidic aqueous media but soluble when the pH is above 5-6. An appropriate polymer is chemically and biologically inert. In order to improve the physical stability of the solid dispersions, the glass transition temperature (Tg) of the polymer is as high as possible. For example, polymers that have a glass transition temperature at least equal to or greater than the glass transition temperature of the API. Other polymers have a glass transition temperature that is within 10 to 15° C. of the API.

Additionally, the hygroscopicity of the polymers is as low, e.g., less than 10%. For the purpose of comparison in this application, the hygroscopicity of a polymer or composition is characterized at 60% relative humidity. In some preferred embodiments, the polymer has less than 10% water absorption, for example less than 9%, less than 8%, less than 7%, less than 6%, less than 5%, less than 4%, less than 3%, or less than 2% water absorption. The hygroscopicity can also affect the physical stability of the solid dispersions. Generally, moisture adsorbed in the polymers can greatly reduce the Tg of the polymers as well as the resulting solid dispersions, which will further reduce the physical stability of the solid dispersions as described above.

In one embodiment, the polymer is one or more water-soluble polymer(s) or partially water-soluble polymer(s). Water-soluble or partially water-soluble polymers include, but are not limited to, cellulose derivatives (e.g., hydroxypropylmethylcellulose (HPMC), hydroxypropylcellulose (HPC)) or ethylcellulose; polyvinylpyrrolidones (PVP); polyethylene glycols (PEG); polyvinyl alcohols (PVA); acrylates, such as polymethacrylate (e.g., Eudragit® E); cyclodextrins (e.g., β-cyclodextin) and copolymers and derivatives thereof, including for example PVP-VA (polyvinylpyrollidone-vinyl acetate).

In some embodiments, the polymer is hydroxypropylmethylcellulose (HPMC), such as HPMC E50, HPMC E15, or HPMC E3.

As discussed herein, the polymer can be a pH-dependent enteric polymer. Such pH-dependent enteric polymers include, but are not limited to, cellulose derivatives (e.g., cellulose acetate phthalate (CAP)), hydroxypropyl methyl cellulose phthalates (HPMCP), hydroxypropyl methyl cellulose acetate succinate (HPMCAS), carboxymethylcellulose (CMC) or a salt thereof (e.g., a sodium salt such as (CMC-Na)); cellulose acetate trimellitate (CAT), hydroxypropylcellulose acetate phthalate (HPCAP), hydroxypropylmethyl-cellulose acetate phthalate (HPMCAP), and methylcellulose acetate phthalate (MCAP), or polymethacrylates (e.g., Eudragit® S). In some embodiments, the polymer is hydroxypropyl methyl cellulose acetate succinate (HPMCAS). In some embodiments, the polymer is hydroxypropyl methyl cellulose acetate succinate HG grade (HPMCAS-HG).

In yet another embodiment, the polymer is a polyvinylpyrrolidone co-polymer, for example, a vinylpyrrolidone/vinyl acetate co-polymer (PVP/VA).

In embodiments where Compound II or Compound III forms a solid dispersion with a polymer, for example with an IPMC, HPMCAS, or PVP/VA polymer, the amount of polymer relative to the total weight of the solid dispersion ranges from 0.1% to 99% by weight. Unless otherwise specified, percentages of drug, polymer and other excipients as described within a dispersion are given in weight percentages. The amount of polymer is typically at least 20%, and preferably at least 30%, for example, at least 35%, at least 40%, at least 45%, or 50% (e.g., 49.5%). The amount is typically 99% or less, and preferably 80% or less, for example 75% or less, 70% or less, 65% or less, 60% or less, or 55% or less. In one embodiment, the polymer is in an amount of up to 50% of the total weight of the dispersion (and even more specifically, between 40% and 50%, such as 49%, 49.5%, or 50%).

In some embodiments, Compound II (or Compound III) and polymer are present in roughly equal amounts in weight, for example each of the polymer and the drug make up half of the percentage weight of the dispersion. For example, the polymer is present in 49.5 wt % and substantially amorphous or amorphous Compound II (or substantially amorphous or amorphous Compound III) is present in 50 wt %. In another embodiment, substantially amorphous or amorphous Compound II (or substantially amorphous or amorphous Compound III) is present in an amount greater than half of the percentage weight of the dispersions. In some embodiments, a first solid dispersion comprises from 70 wt % to 90 wt % (e.g., from 75 wt % to 85 wt %) of substantially amorphous or amorphous Compound II. In some embodiments, a second solid dispersion comprises from 70 wt % to 90 wt % (e.g., from 75 wt % to 85 wt %) of substantially amorphous or amorphous Compound III. For example, in some embodiments, in a solid dispersion comprising Compound II, the polymer is present in 20 wt % and substantially amorphous or amorphous Compound II is present in 80 wt %. In other embodiments, a solid dispersion of substantially amorphous or amorphous Compound III comprises 19.5 wt % of polymer, 80 wt % of substantially amorphous or amorphous Compound III, and 0.5 wt % of SLS.

In one embodiment, the solid dispersion of substantially amorphous or amorphous Compound II comprises about 80 percent of substantially amorphous or amorphous Compound II by weight of the solid dispersion, and about 20 percent of HPMC by weight of the solid dispersion.

In one embodiment, the solid dispersion of substantially amorphous or amorphous Compound III comprises about 80 percent of substantially amorphous or amorphous Compound III by weight of the solid dispersion, and about 19.5 percent of HPMCAS by weight of the solid dispersion, and about 0.5 percent SLS by weight of the dispersion.

Any suitable spray dried dispersion(s) of substantially amorphous or amorphous Compound II and substantially amorphous or amorphous Compound III can be used for the pharmaceutical compositions disclosed herein. Some examples for spray-dried dispersions of substantially amorphous or amorphous Compound II and its pharmaceutically acceptable salts can be found in WO 2015/160787 and WO 2011/119984, the contents of which are incorporated herein by reference. Some examples for spray-dried dispersions of substantially amorphous or amorphous Compound III and its pharmaceutically acceptable salts can be found in WO 2013/130669, WO 2010/019239, and WO 2007/079139, all of which are incorporated herein by reference.

### III. Pharmaceutical Compositions

In one aspect, the present invention provides pharmaceutical compositions comprising an admixture of two CF corrector APIs (e.g., crystalline Compound I Form A and a solid dispersion of substantially amorphous or amorphous Compound II) and a CF potentiator API (e.g., a solid dispersion of substantially amorphous or amorphous Compound III). As exemplified herein, the pharmaceutical composition of the present invention can be a powder admixture of Compound I, Compound II, and Compound III and one or more excipients described herein. Alternatively, the pharmaceutical composition can be formulated into granules, pellets, particles, or one or more mini-tablets. The pharmaceutical composition is capable of being formulated into a unit dosage form, for example, a tablet, capsule, sachet, troches, blister pack and the like containing the powder and/or compressed form of the pharmaceutical composition of the present invention in specified dosage amounts.

In some embodiments, the present invention provides a pharmaceutical composition in the form of a mini-tablet or granule, each granule comprising from about 0.5 to about 1.5 mg of crystalline Compound I Form A. For example, each granule/mini-tablet may comprise about 0.5 mg, about 0.75 mg, about 1 mg, about 1.25 mg, or about 1.5 mg of crystalline Compound I Form A. Each unit dosage form (e.g., package of granules) provides about 20 mg to about 100 mg of crystalline Compound I Form A. In some embodiments, the unit dose form comprises about 20 mg of crystalline Compound I Form A. In some embodiments, the unit dose form comprises about 80 mg of crystalline Compound I Form A. In some embodiments, the unit dose form comprises about 100 mg of crystalline Compound I Form A.

In some embodiments, the present invention provides a pharmaceutical composition in the form of a mini-tablet or granule, each granule comprising from about 0.25 to about 0.75 mg of amorphous or substantially amorphous Compound II. For example, each granule/mini-tablet may comprise about 0.25 mg, about 0.5 mg, or about 0.75 mg of amorphous or substantially amorphous Compound II. In some embodiments, the unit dose form comprises about 10 mg of amorphous or substantially amorphous Compound II. In some embodiments, the unit dose form comprises about 40 mg of amorphous or substantially amorphous Compound II. In some embodiments, the unit dose form comprises about 50 mg of amorphous or substantially amorphous Compound II.

In some embodiments, the present invention provides a pharmaceutical composition in the form of a minitablet or granule, each granule comprising from about 0. 5 to about 1.0 mg of amorphous or substantially amorphous Compound III. For example, each granule/minitablet may comprise about 0.5 mg, about 0.7 mg, about 0.8 mg, or about 1.0 mg of amorphous or substantially amorphous Compound III. In some embodiments, the unit dose form comprises about 15 mg of amorphous or substantially amorphous Compound III. In some embodiments, the unit dose form comprises about 60 mg of amorphous or substantially amorphous Compound III. In some embodiments, the unit dose form comprises about 75 mg of amorphous or substantially amorphous Compound III.

In some embodiments, the present invention provides a pharmaceutical composition in the form of a minitablet or granule, each granule or mini-tablet comprising from about 0.5 to about 1.5 mg of crystalline Compound I Form A, from about 0.25 to about 0.75 mg of amorphous or substantially amorphous Compound II and from about 0. 5 to about 1.0 mg of amorphous or substantially amorphous Compound III. In some embodiments, each granule/mini-tablet comprises 1.1 mg of crystalline Compound I Form A, 0.55 mg of amorphous or substantially amorphous Compound II, and 0.82 mg of amorphous or substantially amorphous Compound III.

In some embodiments, the present invention provides a pharmaceutical composition comprising:
a. crystalline Compound I Form A;
b. a solid dispersion comprising substantially amorphous Compound II and a polymer;
c. a solid dispersion comprising substantially amorphous Compound III and a polymer;
d. one or more fillers;
e. a disintegrant;
f. a sweetener;
g. a glidant; and
h. a lubricant.

In some embodiments, the present invention provides a pharmaceutical composition comprising:
a. crystalline Compound I Form A;
b. a solid dispersion comprising both substantially amorphous Compound II substantially amorphous Compound III;
c. one or more fillers;
xxx. a disintegrant;
d. a sweetener;
e. a glidant; and
f. a lubricant.

In some embodiments, the present invention provides a pharmaceutical composition comprising:
a. crystalline Compound I Form A;
b. a solid dispersion comprising substantially amorphous Compound II and HPMC;
c. a solid dispersion comprising substantially amorphous Compound III and HPMCAS;
d. one or more fillers;
e. a disintegrant;
f. a sweetener;
g. a glidant; and
h. a lubricant.

In some embodiments, the present invention provides a pharmaceutical composition comprising:
a. crystalline Compound I Form A;
b. a solid dispersion containing about 80 wt % of substantially amorphous Compound II and about 20 wt % of HPMC;
c. a solid dispersion containing about 80 wt % of substantially amorphous Compound III, about 19.5 wt % of HPMCAS, and about 0.5 wt % of SLS;
d. one or more fillers;
e. a disintegrant;
f. a sweetener;
g. a glidant; and
h. a lubricant.

In addition to the solid dispersion of Compound II and Compound III, pharmaceutical compositions of the present invention also comprise one or more excipients such as fillers, sweeteners, disintegrants, wetting agents, glidants, lubricants, colorants, flavoring agents or combinations thereof. It is noted that some excipients may serve more than one function, such as some fillers can also be sweeteners and some disintegrants can also be wetting agents (e.g., mannitol is filler and sweetener).

Fillers suitable for the present invention are compatible with the ingredients of the pharmaceutical composition, i.e., they do not substantially reduce the solubility, the chemical stability, the physical stability, or the biological activity of the pharmaceutical composition. Examples of the filler(s) can include, but are not limited to, mannitol, lactose, sucrose, dextrose, maltodextrin, sorbitol, xylitol, powdered cellulose, polyhydric alcohols, microcrystalline cellulose, silicified microcrystalline cellulose, cellulose acetate, methylcellulose, ethylcellulose, hydroxyethylcellulose, methylhydroxyethylcellulose, talc, starch (i.e., potato starch), pregelatinized starch, dibasic calcium phosphate, calcium sulfate, and calcium carbonate, or combinations thereof.

In one embodiment, the filler comprises microcrystalline cellulose.

In a further embodiment, the one or more fillers in the pharmaceutical composition of the invention is a binary filler comprising a mixture of 2 fillers. In another further embodiment, the binary filler is a mixture of mannitol and another filler. In another embodiment, the binary filler is a mixture of lactose, e.g., lactose monohydrate, and another filler. In certain embodiments, the binary filler is a mixture of mannitol and lactose, e.g., lactose monohydrate.

In some embodiments, the pharmaceutical composition comprises a binary filler, wherein the binary filler comprises mannitol and another filler in a ratio of about 3:1 mannitol to other filler, a ratio of about 1:1 mannitol to other filler, or a ratio of about 1:3 mannitol to other filler. In some embodiments, the pharmaceutical composition comprises a binary filler, wherein the binary filler comprises lactose and another filler in a ratio of about 3:1 lactose to other filler, a ratio of about 1:1 lactose to other filler, or a ratio of about 1:3 lactose to other filler. In some embodiments, the pharmaceutical composition comprises a binary filler, wherein the binary filler comprises mannitol and lactose in a ratio of about 3:1 mannitol to lactose or a ratio of about 1:1 mannitol to lactose. In certain embodiments, the binary filler is composed of mannitol and lactose in a ratio of about 1:3 mannitol to lactose.

The pharmaceutical composition also comprises a sweetener to mask and enhance the taste of the composition. In some embodiments, one or more sweeteners include, but are not limited to, monosaccharides, disaccharides and polysaccharides. Examples of suitable sweeteners include both natural and artificial sweeteners. Examples can include, but are not limited to, glucose, sucrose, maltose, mannose, dextrose, fructose, lactose, trehalose, maltitol, lactitol, xylitol, sorbitol, mannitol, tagatose, glycerin, erythritol, isomalt, maltose, sucralose, aspartame, neotame, alitame, neohesperidin dihydrochalcone, cyclamate (i.e. sodium cyclamate), thaumatin, acesulfame potassium, saccharin, and saccharin sodium. In certain embodiments, the sweetener comprises sucralose in a concentration of about 1 wt % to about 2 wt %.

Disintegrants suitable for the present invention enhance the dispersal of the pharmaceutical composition and are compatible with the ingredients of the pharmaceutical composition, i.e., they do not substantially reduce the chemical stability, the physical stability, or the biological activity of the pharmaceutical composition. Exemplary disintegrants include: croscarmellose sodium (e.g., AcDiSol), sodium alginate, calcium alginate, alginic acid, starch, pregelatinized starch, sodium starch glycolate, polyvinylpyrrolidone, co polymers of polyvinylpyrrolidone, crospovidone, carboxymethylcellulose calcium, cellulose and its derivatives, carboxymethylcellulose sodium, soy polysaccharide, clays, gums (i.e. guar gum), an ion exchange resin, an effervescent system based on food acids and an alkaline carbonate component, and sodium bicarbonate. In some embodiments, the pharmaceutical composition comprises about 4 wt % to about 8% of croscarmellose sodium, by weight of the composition. In another example, the pharmaceutical composition comprises about 6 wt % of croscarmellose sodium, by weight of the composition.

Wetting agents and/or surfactants suitable for the present invention can enhance the solubility or the wettability of the pharmaceutical composition and are compatible with the ingredients of the pharmaceutical composition, i.e., they do not substantially reduce the chemical stability, the physical stability, or the biological activity of the pharmaceutical composition. In some embodiments, the one or more wetting agents include one or more surfactants. Examples of wetting agents/surfactants may include, but are not limited to the following: sodium lauryl sulfate (also called sodium dodecyl sulfate (SDS)), cetostearyl alcohol, cetomacrogol emulsifying wax, gelatin, casein, docusate sodium, benzalkonium chloride, calcium stearate, polyethylene glycols, phosphates, polyoxyethylene sorbitan fatty acid esters (e.g. Polysorbate 80, Polysorbate 20), gum acacia, cholesterol, tragacanth, polyoxyethylene 20 stearyl ethers, polyoxyethylene alkyl ethers, polyoxyethylene castor oil derivatives, pegylated hydrogenated castor oils, sorbitan esters of fatty acids, Vitamin E or tocopherol derivatives, vitamin E TPGS, tocopheryl esters, lecithin, phospholipids and their derivatives, poloxamers, stearic acid, oleic acid, oleic alcohol, cetyl alcohol, mono and diglycerides, propylene glycol esters of fatty acids, glycerol esters of fatty acids (i.e. glycerol monostearate), ethylene glycol palmitostearate, polyoxylglycerides, propylene glycol monocaprylate, propylene glycol monolaurate, alkyl aryl polyether alcohols (Triton®) and polyglyceryl oleate. The use of wetting agents in the pharmaceutical compositions of the invention is optional.

Glidants suitable for the present invention enhance the flow properties of the pharmaceutical composition and are compatible with the ingredients of the pharmaceutical composition, i.e., they do not substantially reduce the solubility, the chemical stability, the physical stability, or the biological activity of the pharmaceutical composition. A “glidant” is a substance to promote powder flow by reducing interparticle friction and cohesion. In certain embodiments, the one or more excipients can include one or more glidants. Examples of the glidants may include, but are not limited to, talc, colloidal silica (e.g., Cabosil M-5P), precipitated silica, magnesium oxide, magnesium silicate, leucine and starch. In some embodiments, the compositions of the invention contain colloidal silicon dioxide as a glidant. In some embodiments, the pharmaceutical compositions comprise about 0.5 to about 1.5 wt % of colloidal silicon dioxide, by weight of the composition. In certain embodiments, the pharmaceutical compositions comprise about 1.0 wt % of colloidal silicon dioxide, by weight of the composition.

Lubricants suitable for the present invention improve the compression and ejection of compressed pharmaceutical compositions from a die. Lubricants may further have anti-sticking or anti-tacking properties, and minimize sticking in various operations of the present invention, including operations such as encapsulation, and are compatible with the ingredients of the pharmaceutical composition, i.e., they do not substantially reduce the solubility, or the biological activity of the pharmaceutical composition. Examples of the lubricants may include, but are not limited to, talc, fatty acid, stearic acid, magnesium stearate, calcium stearate, sodium stearate, stearic acid, glyceryl monostearate, sodium lauryl sulfate, sodium stearyl fumarate, hydrogenated oils (i.e., hydrogenated vegetable oil), polyethylene glycol, fatty alcohol, fatty acid ester, glyceryl behenate, mineral oil, vegetable oil, leucine, sodium benzoate, or a combination thereof. In some embodiments, the pharmaceutical compositions of the invention comprise magnesium stearate as a glidant. In some embodiments, the pharmaceutical composition comprises about 0.5 to about 1.5 wt % of magnesium stearate, by weight of the composition. In certain embodiments, the pharmaceutical composition of the invention comprises about 1.0 wt % of magnesium stearate, by weight of the composition.

Pharmaceutical compositions of the present invention can optionally comprise one or more colorants, flavors, and/or fragrances to enhance the visual appeal, taste, and/or scent of the composition. Suitable colorants, flavors, or fragrances are compatible with the ingredients of the pharmaceutical composition, i.e., they do not substantially reduce the solubility, the chemical stability, the physical stability or the biological activity of the pharmaceutical composition. In one embodiment, the pharmaceutical composition comprises a colorant, a flavor, and/or a fragrance.

Suitable flavoring agents can include, for example, flavors, which are known to those of skill in the art, such as, for example, natural flavors, artificial flavors, and combinations thereof. Flavoring agents are compatible with the ingredients of the pharmaceutical composition, i.e., they do not substantially reduce the chemical stability, the physical stability, or the biological activity of the pharmaceutical composition. Flavoring agents may be chosen, e.g., from synthetic flavor oils and flavoring aromatics and/or oils, oleoresins, extracts derived from plants, leaves, flowers, fruits, and the like, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic flower derived or fruit flavors such as vanilla, ethyl vanillin, citrus oils (e.g., lemon, orange, tangerine, lime, and grapefruit), and fruit essences (e.g., natural and/or artificial flavor of apple, pear, peach, orange, grape, strawberry, raspberry, cherry, plum, pineapple, and apricot), and the like, and combinations thereof. The flavoring agents may be used in liquid or solid form and, as indicated above, may be used individually or in admixture. Other flavoring agents can include, for example, certain aldehydes and esters, e.g., cinnamyl acetate, cinnamaldehyde, citral diethylacetal, dihydrocarvyl acetate, eugenyl formate, p-methylamisol, and the like, and combinations thereof.

In some embodiments, the present invention provides a pharmaceutical composition that can be used to treat a patient who possesses mutant forms of human CFTR. In some embodiments, the pharmaceutical composition can include a powder admixture of the pharmaceutical composition ingredients described above formulated to be contained in a capsule, packet, pouch, sachet or some other container operable to provide a unit dose of the powder pharmaceutical composition to a patient in need thereof.

In other embodiments, the present invention provides solid dose forms and unit dose forms comprising a pharmaceutical composition formulated or compressed into a granule, pellet, particle, mini-tablet, sprinkle, and the like. The solid dose forms and unit dose forms comprise compressed powder pharmaceutical compositions as described above with the addition of one or more functional excipients, for example, a disintegrant, glidant, lubricant, filler and/or a wetting agent to facilitate compression of the powder pharmaceutical composition into a compressed pharmaceutical composition, and to facilitate disintegration and dissolution of the compressed powder. The compressed pharmaceutical composition (solid dose forms) such as granules, pellets, particles, mini-tablets and the like can be formulated into unit dose forms such as tablets, capsules, pouches, packets, sachets, bottles and blister packs containing a one or a plurality of such solid dose forms. The number of solid dose forms required for each unit dose form will depend on the concentration of Compounds I, II, and III in each solid dose form (e.g., each granule, pellet or mini-tablet), and the required final amount of Compound I required by the unit dose form. For purposes of illustration only, if a unit dose form (e.g. a capsule, pouch, packet, sachet, bottle or blister pack containing a mini-tablet or plurality of mini-tablets) requires a final dose of about 100 mg of Compound I, 50 mg of Compound II, and 75 mg of Compound III, and each mini-tablet weighs about 7 mg, and each mini-tablet contains about 1.1 mg of Compound I, about 0.55 mg of Compound II, and about 0.82 mg of Compound III, then each capsule or packet should contain about 90 mini-tablets to reach a dose of about 100 mg of Compound I, about 50 mg of Compound II, and about 75 mg of Compound III. If a unit dose form requires a final dose of about 80 mg of Compound I, about 40 mg of Compound II, and about 60 mg of Compound III, and each mini-tablet weighs about 7 mg, then each capsule, pouch, packet, sachet, bottle or blister pack should contain about 72 mini-tablets. If a unit dose form requires a final dose of about 20 mg of Compound I, about 10 mg of Compound II, and about 15 mg of Compound III, and each mini-tablet weighs about 7 mg, then each capsule, pouch, packet, sachet, bottle or blister pack should contain about 18 mini-tablets.

In some embodiments, the present invention provides a pharmaceutical composition comprising a mini-tablet or plurality of mini-tablets in a unit dose form, wherein each of the mini-tablets comprises:
a. crystalline Compound I Form A;
b. a solid dispersion comprising substantially amorphous Compound II and a polymer;
c. a solid dispersion comprising substantially amorphous Compound III and a polymer;
d. one or more fillers;
e. a disintegrant;
f. a sweetener;
g. a glidant; and
h. a lubricant,

wherein the unit dose form comprises crystalline Compound I Form A in an amount ranging from about 20 mg to 100 mg, substantially amorphous Compound II or amorphous Compound II in an amount ranging from about 10 mg to about 50 mg, and substantially amorphous Compound II or amorphous Compound II in an amount ranging from about 15 mg to about 75 mg.

In some embodiments, the present invention provides a pharmaceutical composition comprising a mini-tablet or plurality of mini-tablets in a unit dose form, wherein each of the mini-tablets comprises:
a. crystalline Compound I Form A;
b. a solid dispersion comprising substantially amorphous Compound II and a polymer;
c. a solid dispersion comprising substantially amorphous Compound III and a polymer;
d. one or more fillers;
e. a disintegrant;
f. a sweetener;
g. a glidant; and
h. a lubricant,

wherein the unit dose form comprises 100 mg of crystalline Compound I Form A, 50 mg of substantially amorphous Compound II or amorphous Compound II, and 75 mg of substantially amorphous Compound III or amorphous Compound III.

In some embodiments, the present invention provides a pharmaceutical composition comprising a mini-tablet or plurality of mini-tablets in a unit dose form, wherein each of the mini-tablets comprises:
a. crystalline Compound I Form A;
b. a solid dispersion comprising substantially amorphous Compound II and a polymer;
c. a solid dispersion comprising substantially amorphous Compound III and a polymer;
d. one or more fillers;
e. a disintegrant;
f. a sweetener;
g. a glidant; and
h. a lubricant,

wherein the unit dose form comprises 80 mg of crystalline Compound I Form A, 40 mg of substantially amorphous Compound II or amorphous Compound II, and 60 mg of substantially amorphous Compound III or amorphous Compound III.

In some embodiments, the present invention provides a pharmaceutical composition comprising a mini-tablet or plurality of mini-tablets in a unit dose form, wherein each of the mini-tablets comprises:
a. crystalline Compound I Form A;
b. a solid dispersion comprising substantially amorphous Compound II and a polymer;
c. a solid dispersion comprising substantially amorphous Compound III and a polymer;
d. one or more fillers;
e. a disintegrant;
f. a sweetener;
g. a glidant; and
h. a lubricant,

wherein the unit dose form comprises 20 mg of crystalline Compound I Form A, 10 mg of substantially amorphous Compound II or amorphous Compound II, and 15 mg of substantially amorphous Compound III or amorphous Compound III.

In some embodiments, the present invention provides a pharmaceutical composition comprising a mini-tablet or plurality of mini-tablets in a unit dose form, wherein each of the mini-tablets comprises:
a. crystalline Compound I Form A;
b. a solid dispersion containing about 80 wt % of substantially amorphous Compound II and about 20 wt % of HPMC;
c. a solid dispersion containing about 80 wt % of substantially amorphous Compound III, about 19.5 wt % of HPMCAS, and about 0.5 wt % of SLS;
d. a binary filler composed of mannitol and lactose;
e. croscarmellose sodium;
f. sucralose;
g. colloidal silicon dioxide; and
h. magnesium stearate,

wherein the unit dose form comprises crystalline Compound I Form A in an amount ranging from about 20 mg to 100 mg, substantially amorphous Compound II or amorphous Compound II in an amount ranging from about 10 mg to about 50 mg, and substantially amorphous Compound III or amorphous Compound III in an amount ranging from about 15 mg to about 75 mg.

In some embodiments, the present invention provides a pharmaceutical composition comprising a mini-tablet or plurality of mini-tablets in a unit dose form, wherein each of the mini-tablets comprises:
a. crystalline Compound I Form A;
b. a solid dispersion containing about 80 wt % of substantially amorphous Compound II and about 20 wt % of HPMC;
c. a solid dispersion containing about 80 wt % of substantially amorphous Compound III, about 19.5 wt % of HPMCAS, and about 0.5 wt % of SLS;
d. a binary filler composed of mannitol and lactose;
e. croscarmellose sodium;
f. sucralose;
g. colloidal silicon dioxide; and
h. magnesium stearate.

wherein the unit dose form comprises 100 mg of crystalline Compound I Form A, 50 mg of substantially amorphous Compound II or amorphous Compound II, and 75 mg of substantially amorphous Compound III or amorphous Compound III.

In some embodiments, the present invention provides a pharmaceutical composition comprising a mini-tablet or plurality of mini-tablets in a unit dose form, wherein each of the mini-tablets comprises:
a. crystalline Compound I Form A;
b. a solid dispersion containing about 80 wt % of substantially amorphous Compound II and about 20 wt % of HPMC;
c. a solid dispersion containing about 80 wt % of substantially amorphous Compound III, about 19.5 wt % of HPMCAS, and about 0.5 wt % of SLS;
d. a binary filler composed of mannitol and lactose;
e. croscarmellose sodium;
f. sucralose;
g. colloidal silicon dioxide; and
h. magnesium stearate.

wherein the unit dose form comprises 80 mg of crystalline Compound I Form A, 40 mg of substantially amorphous Compound II or amorphous Compound II, and 60 mg of substantially amorphous Compound III or amorphous Compound III.

In some embodiments, the present invention provides a pharmaceutical composition comprising a mini-tablet or plurality of mini-tablets in a unit dose form, wherein each of the mini-tablets comprises:
a. crystalline Compound I Form A;
b. a solid dispersion containing about 80% by wt of substantially amorphous Compound II and about 20% by weight of HPMC;
c. a solid dispersion containing about 80% by wt of substantially amorphous Compound III, about 19.5 wt % of HPMCAS, and about 0.5 wt % of SLS;
d. a binary filler composed of mannitol and lactose;
e. croscarmellose sodium;
f. sucralose;
g. colloidal silicon dioxide; and
h. magnesium stearate.

wherein the unit dose form comprises 20 mg of crystalline Compound I Form A, 10 mg of substantially amorphous Compound II or amorphous Compound II, and 15 mg of substantially amorphous Compound III or amorphous Compound III.

### IV. Administration of a Pharmaceutical Composition

In another aspect, the invention also provides a method of treating or lessening the severity of a disease in a patient comprising administering to said patient one of the pharmaceutical compositions as defined herein. In some embodiments, the disease is cystic fibrosis.

In some embodiments, the methods of administration of the present invention includes orally administering a liquid or beverage including, but not limited to, milk (including breast milk), baby formula or infant formula, or a soft food including, but not limited to, apple sauce, plain yogurt, ice cream, baby food (including carrots and carrot puree) into which the unit dose form of a pharmaceutical composition of the invention has been sprinkled. It is noted that any of the methods of administration of the present invention can optionally include orally administering with fat-containing food such as a standard CF high-calorie, high-fat meal or snack. In some embodiments, any of the methods of administration of the present invention can optionally include orally administering concurrently with, before or after fat-containing food such as a standard CF high-calorie, high-fat meal or snack.

It is also noted that the methods of administration of the present invention include administering the compositions of the present invention to a patient according to age or weight. In some embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient is 12 to 24 months or 2 to 5 years of age. In still other embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient includes, but not limited to, those weighing greater than or equal to about 14 kilograms. In other embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient includes, but not limited to, those weighing less than 14 kilograms. In still other embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient includes those weighing about 7.5 kilograms to less than 14 kilograms. In other embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient includes to those weighing about 5 kilograms to less than 7.5 kilograms. In other embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient includes those weighing about 2.5 kilograms to less than 5 kilograms. In other embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient includes those weighing less than 7.5 kilograms. In other embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient includes those weighing about 5 kilograms to more than 5 kilograms. In other embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient includes those weighing less than 5 kilograms. In other embodiments, the method of administering a pharmaceutical composition includes orally administering to a patient every 12 hours at least one capsule or packet containing a mini-tablet or plurality of mini-tablets, wherein each mini-tablet comprising a pharmaceutical composition as described herein, wherein the patient includes those weighing about 2.5 kilograms to more than 2.5 kilograms.

It is also noted that the methods of administration of the present invention include administering the compositions of the present invention to a patient according to age or weight. In some embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient is 12 to 24 months or 2 to 5 years of age. In still other embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient includes, but not limited to, those weighing greater than or equal to about 14 kilograms. In other embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient includes, but not limited to, those weighing less than 14 kilograms. In still other embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient includes those weighing about 7.5 kilograms to less than 14 kilograms. In other embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient includes to those weighing about 5 kilograms to less than 7.5 kilograms. In other embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient includes those weighing about 2.5 kilograms to less than 5 kilograms. In other embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient includes those weighing less than 7.5 kilograms. In other embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient includes those weighing about 5 kilograms to more than 5 kilograms. In other embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient includes those weighing less than 5 kilograms. In other embodiments, the method of administering a pharmaceutical composition includes orally administering to a patient once a day at least one capsule or packet containing a mini-tablet or plurality of mini-tablets, wherein each mini-tablet comprising a pharmaceutical composition as described herein, wherein the patient includes those weighing about 2.5 kilograms to more than 2.5 kilograms.

It is also noted that the methods of administration of the present invention include administering the compositions of the present invention to a patient according to age or weight. In some embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient is 12 to 24 months or 2 to 5 years of age. In still other embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient includes, but not limited to, those weighing greater than or equal to about 14 kilograms. In other embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient includes, but not limited to, those weighing less than 14 kilograms. In still other embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient includes those weighing about 7.5 kilograms to less than 14 kilograms. In other embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient includes to those weighing about 5 kilograms to less than 7.5 kilograms. In other embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient includes those weighing about 2.5 kilograms to less than 5 kilograms. In other embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient includes those weighing less than 7.5 kilograms. In other embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient includes those weighing about 5 kilograms to more than 5 kilograms. In other embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient includes those weighing less than 5 kilograms. In other embodiments, the method of administering a pharmaceutical composition includes orally administering to a patient twice a day at least one capsule or packet containing a mini-tablet or plurality of mini-tablets, wherein each mini-tablet comprising a pharmaceutical composition as described herein, wherein the patient includes those weighing about 2.5 kilograms to more than 2.5 kilograms.

In some embodiments, the method includes treating or lessening the severity of cystic fibrosis in a patient comprising administering to said patient a pharmaceutical composition comprising a powder composition or a compressed pharmaceutical composition. In one embodiment, a capsule or a packet containing a powder pharmaceutical composition comprising about 20 mg to about 100 mg of Compound I, about 10 mg to about 50 mg of amorphous or substantially amorphous Compound II, and about 15 mg to about 75 mg of amorphous or substantially amorphous Compound III is administered to the patient.

In some embodiments, the method includes treating or lessening the severity of cystic fibrosis in a patient comprising administering to said patient a compressed pharmaceutical composition of the invention, i.e., a capsule or a packet containing minitablets or granules comprising about 100 mg of Compound I, about 50 mg of Compound II and about 75 mg of Compound III. In some embodiments, the method includes treating or lessening the severity of cystic fibrosis in a patient comprising administering to said patient a compressed pharmaceutical composition of the invention, i.e., a capsule or a packet containing minitablets or granules comprising about 80 mg of Compound I, about 40 mg of Compound II and about 60 mg of Compound III. In some embodiments, the method includes treating or lessening the severity of cystic fibrosis in a patient comprising administering to said patient a compressed pharmaceutical composition of the invention, i.e., a capsule or a packet containing minitablets or granules comprising about 20 mg of Compound I, about 10 mg of Compound II and about 15 mg of Compound III.

It is also noted that the methods of administration of the present invention can optionally include orally administering a pharmaceutical composition as described herein in the absence of food or beverage. In the present method, the oral administration is performed directly after, or shortly after (e.g., within 30 minutes) the patient eats or drinks. In another embodiment, the oral administration is performed at least 1 hour (e.g., at least 2 hours, at least 3 hours, at least 4 hours, at least 5 hours, at least 8 hours, at least 12 hours or at least 24 hours) after eating or drinking. For instance, in one example, the method of administering a pharmaceutical composition includes orally administering to a patient at least once per day at least one capsule or the contents of a packet or a pouch comprising a mini-tablet or plurality of mini-tablets, each mini-tablet containing Compound I, Compound II, and Compound III, one or more fillers, a sweetener, a disintegrant, optionally a wetting agent, a glidant; and a lubricant, and another therapeutic or medical procedure. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.”

In one embodiment, the additional therapeutic agent is selected from a mucolytic agent, bronchodilator, an anti-biotic, an anti-infective agent, an anti-inflammatory agent, or a nutritional agent.

In one embodiment, the additional agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levofloxacin, including aerosolized formulations thereof, and combinations of two antibiotics, e.g., fosfomycin and tobramycin.

In another embodiment, the additional agent is a mucolyte. Exemplary mucolytes useful herein includes Pulmozyme®.

In another embodiment, the additional agent is a bronchodilator. Exemplary bronchodilators include albuterol, metaproterenol sulfate, pirbuterol acetate, salmeterol, or terbutaline sulfate.

In another embodiment, the additional agent is effective in restoring lung airway surface liquid. Such agents improve the movement of salt in and out of cells, allowing mucus in the lung airway to be more hydrated and, therefore, cleared more easily. Exemplary such agents include hypertonic saline, denufosol tetrasodium ([[(3S,5R)-5-(4-amino-2-oxopyrimidin-1-yl)-3-hydroxyoxolan-2-yl]methoxy-hydroxyphosphoryl][[[(2R,3S,4R,5R)-5-(2,4-dioxopyrimidin-1-yl)-3,4-dihydroxyoxolan-2-yl]methoxy-hydroxyphosphoryl]oxy-hydroxyphosphoryl]hydrogen phosphate), or bronchitol (inhaled formulation of mannitol).

In another embodiment, the additional agent is an anti-inflammatory agent, i.e., an agent that can reduce the inflammation in the lungs. Exemplary such agents useful herein include ibuprofen, docosahexaenoic acid (DHA), sildenafil, inhaled glutathione, pioglitazone, hydroxychloroquine, or simavastatin.

In another embodiment, the additional agent is a CFTR modulator other than Compound I, i.e., an agent that has the effect of modulating CFTR activity. Exemplary such agents include ataluren (“PTC124®”; 3-[5-(2-fluorophenyl)-1,2,4-oxadiazol-3-yl]benzoic acid), sinapultide, lancovutide, depelestat (a human recombinant neutrophil elastase inhibitor), cobiprostone (7-{(2R, 4aR, 5R, 7aR)-2-[(3S)-1,1-difluoro-3-methylpentyl]-2-hydroxy-6-oxooctahydrocyclopenta[b]pyran-5-yl}heptanoic acid), or (3-(6-(1-(2,2-difluorobenzo[d][1,3]dioxol-5-yl) cyclopropanecarboxamido)-3-methylpyridin-2-yl)benzoic acid. In another embodiment, the additional agent is (3-(6-(1-(2,2-difluorobenzo[d][1,3]dioxol-5-yl) cyclopropanecarboxamido)-3-methylpyridin-2-yl)benzoic acid.

In another embodiment, the additional agent is a nutritional agent. Exemplary such agents include pancrelipase (pancreating enzyme replacement), including Pancrease®, Pancreacarb®, Ultrase®, or Creon®, Liprotamase® (formerly Trizytek®), Aquadeks®, or glutathione inhalation. In one embodiment, the additional nutritional agent is pancrelipase.

### V. EXAMPLES

In order that the invention described herein may be more fully understood, the following examples are set forth. It should be understood that these examples are for illustrative purposes only and are not to be construed as limiting this invention in any manner.

### Example 1: Synthesis of Compound I: N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound I)

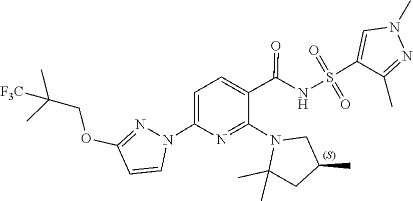

The syntheses of Compound I and crystalline Compound I Form A are fully described in WO 2018/107100 and WO 2019/018395, the contents of which are incorporated herein by reference. These references demonstrate that Compound I is a potent CFTR corrector therapeutic.

Part A: Synthesis of (4S)-2,2,4-trimethylpyrrolidine hydrochloride

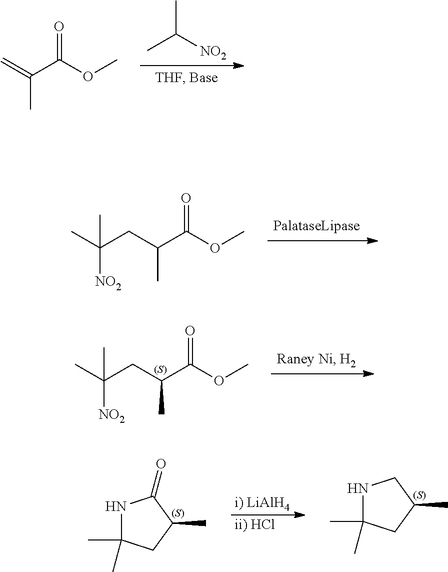

### Step 1: methyl-2,4-dimethyl-4-nitro-pentanoate

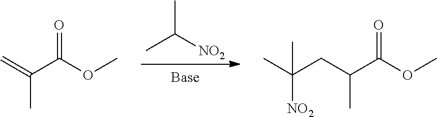

Tetrahydrofuran (THF, 4.5 L) was added to a 20 L glass reactor and stirred under N₂ at room temperature. 2-Nitropropane (1.5 kg, 16.83 mol) and 1,8-diazabicyclo[5.4.0]undec-7-ene (DBU) (1.282 kg, 8.42 mol) were then charged to the reactor, and the jacket temperature was increased to 50° C. Once the reactor contents were close to 50° C., methyl methacrylate (1.854 kg, 18.52 mol) was added slowly over 100 minutes. The reaction temperature was maintained at or close to 50° C. for 21 hours. The reaction mixture was concentrated in vacuo then transferred back to the reactor and diluted with methyl tert-butyl ether (MTBE) (14 L). 2 M HCl (7.5 L) was added, and this mixture was stirred for 5 minutes then allowed to settle. Two clear layers were visible—a lower yellow aqueous phase and an upper green organic phase. The aqueous layer was removed, and the organic layer was stirred again with 2 M HCl (3 L). After separation, the HCl washes were recombined and stirred with MTBE (3 L) for 5 minutes. The aqueous layer was removed, and all of the organic layers were combined in the reactor and stirred with water (3 L) for 5 minutes. After separation, the organic layers were concentrated in vacuo to afford a cloudy green oil. Crude product was treated with MgSO₄ and filtered to afford methyl-2,4-dimethyl-4-nitro-pentanoate as a clear green oil (3.16 kg, 99% yield).

¹H NMR (400 MHz, Chloroform-d) δ 3.68 (s, 3H), 2.56-2.35 (m, 2H), 2.11-2.00 (m, 1H), 1.57 (s, 3H), 1.55 (s, 3H), 1.19 (d, J=6.8 Hz, 3H).

### Step 2: Synthesis of methyl (2S)-2,4-dimethyl-4-nitro-pentanoate

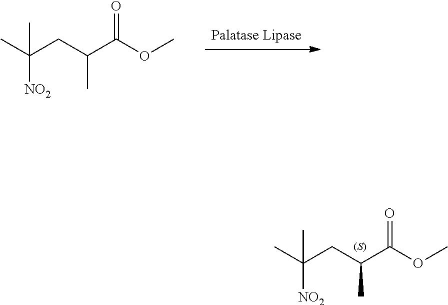

A reactor was charged with purified water (2090 L; 10 vol) and then potassium phosphate monobasic (27 kg, 198.4 moles; 13 g/L for water charge). The pH of the reactor contents was adjusted to pH 6.5 (±0.2) with 20% (w/v) potassium carbonate solution. The reactor was charged with racemic methyl-2,4-dimethyl-4-nitro-pentanoate (209 kg; 1104.6 moles), and Palatase 20000L lipase (13 L, 15.8 kg; 0.06 vol).

The reaction mixture was adjusted to 32±2° C. and stirred for 15-21 hours, and pH 6.5 was maintained using a pH stat with the automatic addition of 20% potassium carbonate solution. When the racemic starting material was converted to >98% ee of the S-enantiomer, as determined by chiral GC, external heating was switched off. The reactor was then charged with MTBE (35 L; 5 vol), and the aqueous layer was extracted with MTBE (3 times, 400-1000L). The combined organic extracts were washed with aqueous Na₂CO₃ (4 times, 522 L, 18% w/w 2.5 vol), water (523 L; 2.5 vol), and 10% aqueous NaCl (314 L, 1.5 vol). The organic layer was concentrated in vacuo to afford methyl (2S)-2,4-dimethyl-4-nitro-pentanoate as a mobile yellow oil (>98% ee, 94.4 kg; 45% yield).

### Step 3: Synthesis of (3S)-3,5,5-trimethylpyrrolidin-2-one

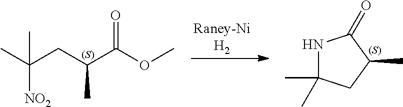

A 20 L reactor was purged with N₂. The vessel was charged sequentially with DI water-rinsed, damp Raney® Ni (2800 grade, 250 g), methyl (2S)-2,4-dimethyl-4-nitro-pentanoate (1741 g, 9.2 mol), and ethanol (13.9 L, 8 vol). The reaction was stirred at 900 rpm, and the reactor was flushed with H₂ and maintained at ˜2.5 bar. The reaction mixture was then warmed to 60° C. for 5 hours. The reaction mixture was cooled and filtered to remove Raney nickel, and the solid cake was rinsed with ethanol (3.5 L, 2 vol). The ethanolic solution of the product was combined with a second equal sized batch and concentrated in vacuo to reduce to a minimum volume of ethanol (˜1.5 volumes). Heptane (2.5 L) was added, and the suspension was concentrated again to ˜1.5 volumes. This was repeated 3 times; the resulting suspension was cooled to 0-5° C., filtered under suction, and washed with heptane (2.5 L). The product was dried under vacuum for 20 minutes then transferred to drying trays and dried in a vacuum oven at 40° C. overnight to afford (3S)-3,5,5-trimethylpyrrolidin-2-one as a white crystalline solid (2.042 kg, 16.1 mol, 87%). ¹H NMR (400 MHz, Chloroform-d) δ 6.39 (s, 1H), 2.62 (ddq, J=9.9, 8.6, 7.1 Hz, 1H), 2.17 (dd, J=12.4, 8.6 Hz, 1H), 1.56 (dd, J=12.5, 9.9 Hz, 1H), 1.31 (s, 3H), 1.25 (s, 3H), 1.20 (d, J=7.1 Hz, 3H).

### Step 4: Synthesis of (4S)-2,2,4-trimethylpyrrolidine hydrochloride

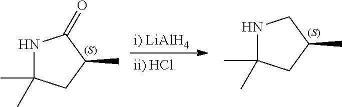

A glass lined 120 L reactor was charged with lithium aluminum hydride pellets (2.5 kg, 66 mol) and dry THE (60 L) and warmed to 30° C. The resulting suspension was charged with (S)-3,5,5-trimethylpyrrolidin-2-one (7.0 kg, 54 mol) in THE (25 L) over 2 hours while maintaining the reaction temperature at 30 to 40° C. After complete addition, the reaction temperature was increased to 60-63° C. and maintained overnight. The reaction mixture was cooled to 22° C., then cautiously quenched with the addition of ethyl acetate (EtOAc) (1.0 L, 10 moles), followed by a mixture of THF (3.4 L) and water (2.5 kg, 2.0 eq), and then a mixture of water (1.75 kg) with 50% aqueous sodium hydroxide (750 g, 2 equiv water with 1.4 equiv sodium hydroxide relative to aluminum), followed by 7.5 L water. After the addition was complete, the reaction mixture was cooled to room temperature, and the solid was removed by filtration and washed with THE (3×25 L). The filtrate and washings were combined and treated with 5.0 L (58 moles) of aqueous 37% HCl (1.05 equiv.) while maintaining the temperature below 30° C. The resultant solution was concentrated by vacuum distillation to a slurry. Isopropanol (8 L) was added and the solution was concentrated to near dryness by vacuum distillation. Isopropanol (4 L) was added, and the product was slurried by warming to about 50° C. MTBE (6 L) was added, and the slurry was cooled to 2-5° C. The product was collected by filtration and rinsed with 12 L MTBE and dried in a vacuum oven (55° C./300 torr/N₂ bleed) to afford (4S)-2,2,4-trimethylpyrrolidine•HCl as a white, crystalline solid (6.21 kg, 75% yield). ¹H NMR (400 MHz, DMSO-d6) δ 9.34 (br d, 2H), 3.33 (dd, J=11.4, 8.4 Hz, 1H), 2.75 (dd, J=11.4, 8.6 Hz, 1H), 2.50-2.39 (m, 1H), 1.97 (dd, J=12.7, 7.7 Hz, 1H), 1.42 (s, 3H), 1.38 (dd, J=12.8, 10.1 Hz, 1H), 1.31 (s, 3H), 1.05 (d, J=6.6 Hz, 3H).

### Part B: Preparation of N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound I)

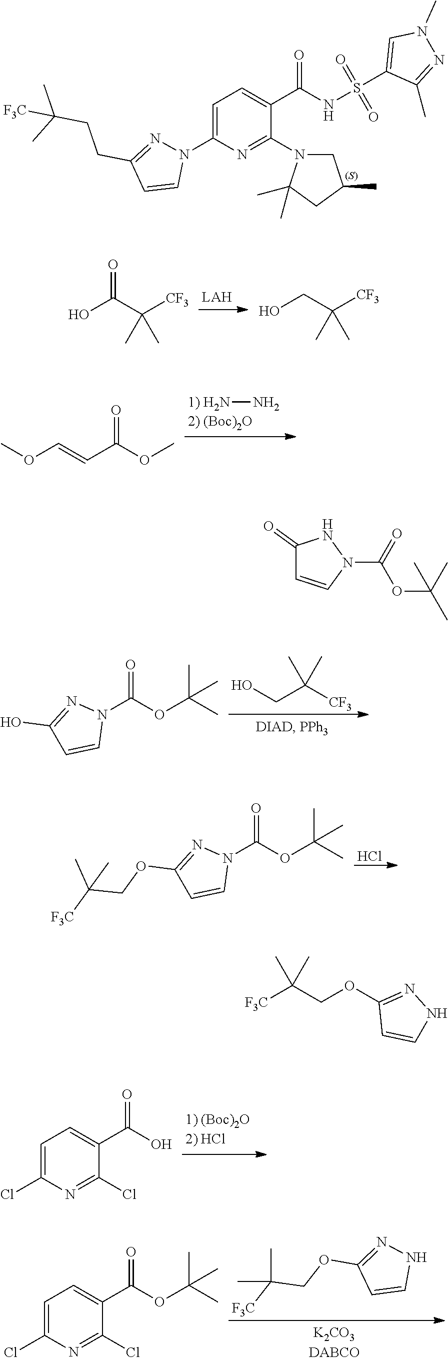

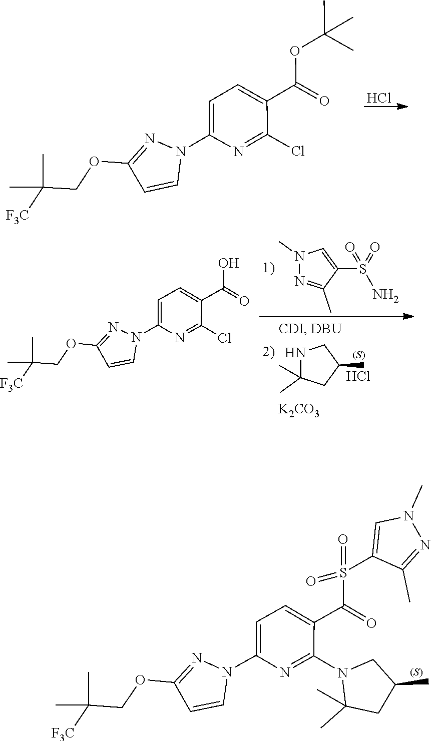

### Preparation of starting materials

### 3,3,3-Trifluoro-2,2-dimethyl-propan-1-ol

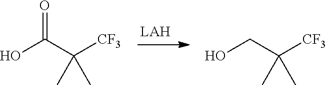

A 1 L 3 neck round bottom flask was fitted with a mechanical stirrer, a cooling bath, an addition funnel, and a J-Kem temperature probe. The vessel was charged with lithium aluminum hydride (LAH) pellets (6.3 g, 0.1665 mol) under a nitrogen atmosphere. The vessel was then charged with tetrahydrofuran (200 mL) under a nitrogen atmosphere. The mixture was allowed to stir at room temperature for 0.5 hours to allow the pellets to dissolve. The cooling bath was then charged with crushed ice in water and the reaction temperature was lowered to 0° C. The addition funnel was charged with a solution of 3,3,3-trifluoro-2,2-dimethyl-propanoic acid (20 g, 0.1281 mol) in tetrahydrofuran (60 mL) and the clear pale yellow solution was added drop wise over 1 hour. After the addition was complete the mixture was allowed to slowly warm to room temperature and stirring was continued for 24 hours. The suspension was cooled to 0° C. with a crushed ice-water in the cooling bath and then quenched by the very slow and drop wise addition of water (6.3 ml), followed by sodium hydroxide solution (15 weight %; 6.3 mL) and then finally with water (18.9 mL). The reaction temperature of the resulting white suspension was recorded at 5° C. The suspension was stirred at −5° C. for 30 minutes and then filtered through a 20 mm layer of Celite. The filter cake was washed with tetrahydrofuran (2×100 mL). The filtrate was dried over sodium sulfate (150 g) and then filtered. The filtrate was concentrated under reduced pressure to provide a clear colorless oil (15 g) containing a mixture of the product 3,3,3-trifluoro-2,2-dimethyl-propan-1-ol in THE (73% weight of product ˜10.95g, and 27 wt. % THE as determined by 1H-NMR). The distillate from the rotary evaporation was distilled at atmospheric pressure using a 30 cm Vigreux column to provide 8.75 g of a residue containing 60% weight of THF and 40% weight of product (˜3.5 g). The estimated total amount of product is 14.45 g (79% yield). 1H NMR (400 MHz, DMSO-d6) δ 4.99 (t, J=5.7 Hz, 1H), 3.38 (dd, J=5.8, 0.9 Hz, 2H), 1.04 (d, J=0.9 Hz, 6H).

### tert-Butyl 3-oxo-2,3-dihydro-1H-pyrazole-1-carboxylate

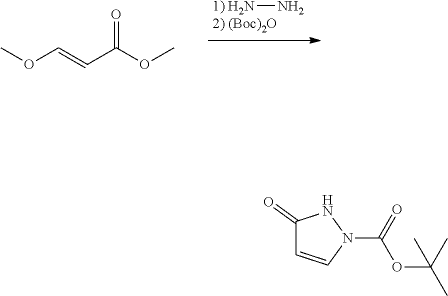

A 50L Syrris controlled reactor was started and jacket set to 20° C., stirring at 150 rpm, reflux condenser (10° C.) and nitrogen purge. MeOH (2.860 L) and methyl (E)-3-methoxyprop-2-enoate (2.643 kg, 22.76 mol) were added and the reactor was capped. The reaction was heated to an internal temperature of 40° C. and the system was set to hold jacket temp at 40° C. Hydrazine hydrate (1300 g of 55% w/w, 22.31 mol) was added portion wise via addition funnel over 30 min. The reaction was heated to 60° C. for 1 h. The reaction mixture was cooled to 20° C. and triethyamine (2.483 kg, 3.420 L, 24.54 mol) was added portion wise (exothermic), maintaining reaction temp <30° C. A solution of Boc anhydride (di-tert-butyl dicarbonate) (4.967 kg, 5.228 L, 22.76 mol) in MeOH (2.860 L) was added portion wise maintaining temperature <45° C. The reaction mixture was stirred at 20° C. for 16 h. The reaction solution was partially concentrated to remove MeOH, resulting in a clear light amber oil. The resulting oil was transferred to the 50L reactor, stirred and added water (7.150 L) and heptane (7.150 L). The additions caused a small amount of the product to precipitate. The aqueous layer was drained into a clean container and the interface and heptane layer were filtered to separate the solid (product). The aqueous layer was transferred back to the reactor, and the collected solid was placed back into the reactor and mixed with the aqueous layer. A dropping funnel was added to the reactor and loaded with acetic acid (1.474 kg, 1.396 L, 24.54 mol), then began dropwise addition of acid. The jacket was set to 0° C. to absorb the quench exotherm. After addition (pH=5), the reaction mixture was stirred for 1 h. The solid was collected by filtration and washed with water (7.150 L), and washed a second time with water (3.575 L) and pulled dry. The crystalline solid was scooped out of the filter into a 20L rotovap bulb and heptane (7.150 L) was added. The mixture was slurried at 45° C. for 30 mins, and then distilled off 1-2 volumes of solvent. The slurry in the rotovap flask was filtered and the solids washed with heptane (3.575 L) and pulled dry. The solid was further dried in vacuo (50° C., 15 mbar) to give tert-butyl 5-oxo-1H-pyrazole-2-carboxylate (2921 g, 71%) as coarse, crystalline solid. ¹H NMR (400 MHz, DMSO-d6) δ 10.95 (s, 1H), 7.98 (d, J=2.9 Hz, 1H), 5.90 (d, J=2.9 Hz, 1H), 1.54 (s, 9H).

### Step A: tert-Butyl 3-(3,3,3-trifluoro-2,2-dimethyl-propoxy)pyrazole-1-carboxylate

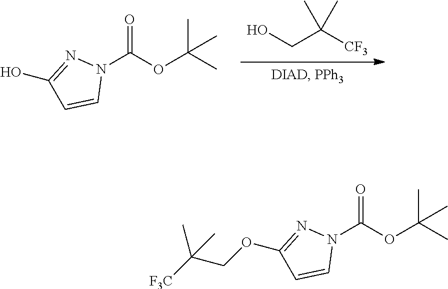

A mixture of 3,3,3-trifluoro-2,2-dimethyl-propan-1-ol (10 g, 70.36 mmol) and tert-butyl 3-hydroxypyrazole-1-carboxylate (12.96 g, 70.36 mmol) in toluene (130 mL) was treated with triphenyl phosphine (20.30 g, 77.40 mmol) followed by isopropyl N-isopropoxycarbonyliminocarbamate (14.99 mL, 77.40 mmol) and the mixture was stirred at 110° C. for 16 hours. The yellow solution was concentrated under reduced pressure, diluted with heptane (100 mL) and the precipitated triphenylphosphine oxide was removed by filtration and washed with heptane/toluene 4:1 (100 mL). The yellow filtrate was evaporated and the residue purified by silica gel chromatography with a linear gradient of ethyl acetate in hexane (0-40%) to give tert-butyl 3-(3,3,3-trifluoro-2,2-dimethyl-propoxy)pyrazole-1-carboxylate (12.3 g, 57%) as an off white solid. ESI-MS m z calc. 308.13477, found 309.0 (M+1)⁺; Retention time: 1.84 minutes. ¹H NMR (400 MHz, DMSO-d6) δ 8.10 (d, J=3.0 Hz, 1H), 6.15 (d, J=3.0 Hz, 1H), 4.18 (s, 2H), 1.55 (s, 9H), 1.21 (s, 6H).

### Step B: 3-(3,3,3-trifluoro-2,2-dimethyl-propoxy)-1H-pyrazole

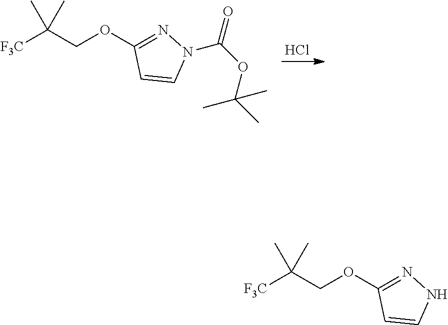 [SMILES: CC(C)(C)OC(=O)n1ccc(OCC(C)(C)C(F)(F)F)n1 ; Cl ; CC(C)(COc1cc[nH]n1)C(F)(F)F]

tert-Butyl 3-(3,3,3-trifluoro-2,2-dimethyl-propoxy)pyrazole-1-carboxylate (13.5 g, 43.79 mmol) was treated with 4 M hydrogen chloride in dioxane (54.75 mL, 219.0 mmol) and the mixture was stirred at 45° C. for 1 hour. The reaction mixture was evaporated to dryness and the residue was extracted with 1 M aqueous NaOH (100 ml) and methyl tert-butyl ether (100 ml), washed with brine (50 ml) and extracted with methyl tert-butyl ether (50 ml). The combined organic phases were dried, filtered and evaporated to give 3-(3,3,3-trifluoro-2,2-dimethyl-propoxy)-1H-pyrazole (9.0 g, 96%) as an off white waxy solid. ESI-MS m z calc. 208.08235, found 209.0 (M+1)⁺; Retention time: 1.22 minutes. ¹H NMR (400 MHz, DMSO-d6) δ 11.91 (s, 1H), 7.52 (d, J=2.2 Hz, 1H), 5.69 (t, J=2.3 Hz, 1H), 4.06 (s, 2H), 1.19 (s, 6H).

### Step C: tert-Butyl 2,6-dichloropyridine-3-carboxylate

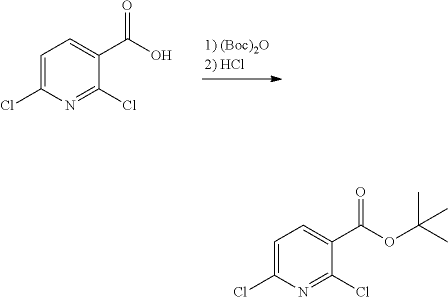

A solution of 2,6-dichloropyridine-3-carboxylic acid (10 g, 52.08 mmol) in THE (210 mL) was treated successively with di-tert-butyl dicarbonate (17 g, 77.89 mmol) and 4-(dimethylamino)pyridine (3.2 g, 26.19 mmol) and left to stir overnight at room temperature. At this point, HCl 1N (400 mL) was added and the mixture was stirred vigorously for about 10 minutes. The product was extracted with ethyl acetate (2×300 mL) and the combined organics layers were washed with water (300 mL) and brine (150 mL) and dried over sodium sulfate and concentrated under reduced pressure to give 12.94 g (96% yield) of tert-butyl 2,6-dichloropyridine-3-carboxylate as a colorless oil. ESI-MS m z calc. 247.01668, found 248.1 (M+1)⁺; Retention time: 2.27 minutes. ¹H NMR (300 MHz, CDCl₃) ppm 1.60 (s, 9H), 7.30 (d, J=7.9 Hz, 1H), 8.05 (d, J=8.2 Hz, 1H).

### Step D: tert-Butyl 2-chloro-6-[3-(3,3,3-trifluoro-2,2-dimethyl-propoxy)pyrazol-1-yl]pyridine-3-carboxylate

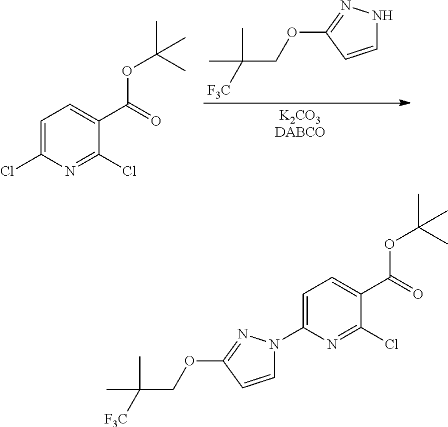

To a solution of tert-butyl 2,6-dichloropyridine-3-carboxylate (10.4 g, 41.9 mmol) and 3-(3,3,3-trifluoro-2,2-dimethyl-propoxy)-1H-pyrazole (9.0 g, 41.93 mmol) in DMF (110 mL) were added potassium carbonate (7.53 g, 54.5 mmol) and 1,4-diazabicyclo[2.2.2]octane (706 mg, 6.29 mmol) and he mixture was stirred at room temperature for 16 hours. The cream suspension was cooled in a cold water bath and cold water (130 mL) was slowly added. The thick suspension was stirred at room temperature for 1 hour, filtered and washed with plenty of water to give tert-butyl 2-chloro-6-[3-(3,3,3-trifluoro-2,2-dimethyl-propoxy)pyrazol-1-yl]pyridine-3-carboxylate (17.6 g, 99%) as an off white solid. ESI-MS m z calc. 419.12234, found 420.0 (M+1)⁺; Retention time: 2.36 minutes. ¹H NMR (400 MHz, DMSO-d6) δ 8.44 (d, J=2.9 Hz, 1H), 8.31 (d, J=8.4 Hz, 1H), 7.76 (d, J=8.4 Hz, 1H), 6.26 (d, J=2.9 Hz, 1H), 4.27 (s, 2H), 1.57 (s, 9H), 1.24 (s, 6H).

Step E: 2-chloro-6-[3-(3,3,3-trifluoro-2,2-dimethyl-propoxy)pyrazol-1-yl]pyridine-3-carboxylic acid

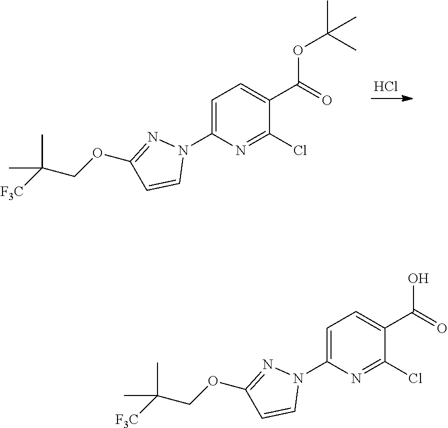

tert-Butyl 2-chloro-6-[3-(3,3,3-trifluoro-2,2-dimethyl-propoxy)pyrazol-1-yl]pyridine-3-carboxylate (17.6 g, 40.25 mmol) was suspended in isopropanol (85 mL) treated with hydrochloric acid (34 mL of 6 M, 201 mmol) and heated to reflux for 3 hours (went almost complete into solution at reflux and started to precipitate again). The suspension was diluted with water (51 mL) at reflux and left to cool to room temperature under stirring for 2.5 h. The solid was collected by filtration, washed with isopropanol/water 1:1 (50 mL), plenty of water and dried in a drying cabinet under vacuum at 45-50° C. with a nitrogen bleed overnight to give 2-chloro-6-[3-(3,3,3-trifluoro-2,2-dimethyl-propoxy)pyrazol-1-yl]pyridine-3-carboxylic acid (13.7 g, 91%) as an off white solid. ESI-MS m z calc. 363.05975, found 364.0 (M+1)⁺; Retention time: 1.79 minutes. ¹H NMR (400 MHz, DMSO-d6) δ 13.61 (s, 1H), 8.44 (d, J=2.9 Hz, 1H), 8.39 (d, J=8.4 Hz, 1H), 7.77 (d, J=8.4 Hz, 1H), 6.25 (d, J=2.9 Hz, 1H), 4.28 (s, 2H), 1.24 (s, 6H).

### Step F: 2-Chloro-N-(1,3-dimethylpyrazol-4-yl)sulfonyl-6-[3-(3,3,3-trifluoro-2,2-dimethyl-propoxy)pyrazol-1-yl]pyridine-3-carboxamide

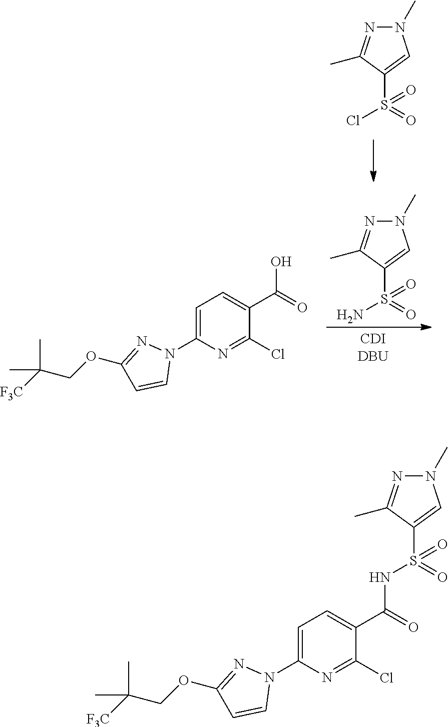

2-Chloro-6-[3-(3,3,3-trifluoro-2,2-dimethyl-propoxy)pyrazol-1-yl]pyridine-3-carboxylic acid (100 mg, 0.2667 mmol) and CDI (512 mg, 3.158 mmol) were combined in THE (582.0 μL) and the mixture was stirred at room temperature. Meanwhile, 1,3-dimethylpyrazole-4-sulfonyl chloride (62 mg, 0.3185 mmol) was combined with ammonia (in methanol) in a separate vial, instantly forming a white solid. After stirring for an additional 20 min, the volatiles were removed by evaporation, and 1 mL of dichloromethane was added to the solid residue, and was also evaporated. DBU (100 μL, 0.6687 mmol) was then added and the mixture stirred at 60° C. for 5 minutes, followed by addition of THF (1 mL) which was subsequently evaporated. The contents of the vial containing the CDI activated carboxylic acid in THE were then added to the vial containing the newly formed sulfonamide and DBU, and the reaction mixture was stirred for 4 hours at room temperature. The reaction mixture was diluted with 10 mL of ethyl acetate, and washed with 10 mL solution of citric acid (1 M). The aqueous layer was extracted with ethyl acetate (2×10 mL) and the combined organics were washed with brine, dried over sodium sulfate, and concentrated to give the product as white solid (137 mg, 99%) that was used in the next step without further purification. ESI-MS m/z calc. 520.09076, found 521.1 (M+1)⁺; Retention time: 0.68 minutes.

### Step G: N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide

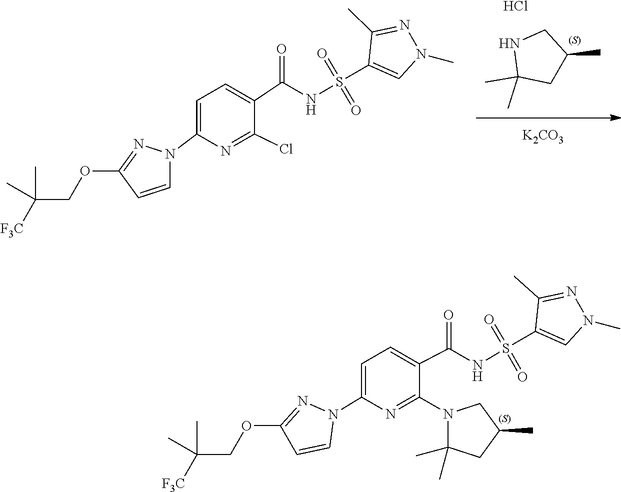

2-Chloro-N-(1,3-dimethylpyrazol-4-yl)sulfonyl-6-[3-(3,3,3-trifluoro-2,2-dimethyl-propoxy)pyrazol-1-yl]pyridine-3-carboxamide (137 mg, 0.2630 mmol), (4S)-2,2,4-trimethylpyrrolidine (Hydrochloride salt) (118 mg, 0.7884 mmol), and potassium carbonate (219 mg, 1.585 mmol) were combined in DMSO (685.0 μL) and the mixture was heated at 130° C. for 16 hours. The reaction was cooled to room temperature, and 1 mL of water was added. After stirring for 15 minutes, the contents of the vial were allowed to settle, and the liquid portion was removed via pipet and the remaining solids were dissolved with 20 mL of ethyl acetate and were washed with 1 M citric acid (15 mL). The layers were separated and the aqueous layer was extracted two additional times with 15 mL of ethyl acetate. The organics were combined, washed with brine, dried over sodium sulfate and concentrated. The resulting solid was further purified by silica gel chromatography eluting with a gradient of methanol in dichloromethane (0-10%) to give N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (72 mg, 41%) as a white solid. ESI-MS m/z calc. 597.2345, found 598.3 (M+1)⁺; Retention time: 2.1 minutes. ¹H NMR (400 MHz, DMSO) δ 12.36 (s, 1H), 8.37 (s, 1H), 8.22 (d, J=2.8 Hz, 1H), 7.74 (d, J=8.2 Hz, 1H), 6.93 (d, J=8.2 Hz, 1H), 6.17 (d, J=2.8 Hz, 1H), 4.23 (s, 2H), 3.81 (s, 3H), 2.56 (d, J=10.4 Hz, 1H), 2.41 (t, J=8.7 Hz, 1H), 2.32 (s, 3H), 2.18 (dd, J=12.4, 6.1 Hz, 1H), 1.87 (dd, J=11.7, 5.5 Hz, 1H), 1.55 (d, J=11.2 Hz, 6H), 1.42 (t, J=12.0 Hz, 1H), 1.23 (s, 6H), 0.81 (d, J=6.2 Hz, 3H).

### Alternative Steps F and G

### Alternative Step F: 2-chloro-N-((1,3-dimethyl-1H-pyrazol-4-yl)sulfonyl)-6-(3-(3,3,3-trifluoro-2,2-dimethylpropoxy)-1H-pyrazol-1-yl)nicotinamide

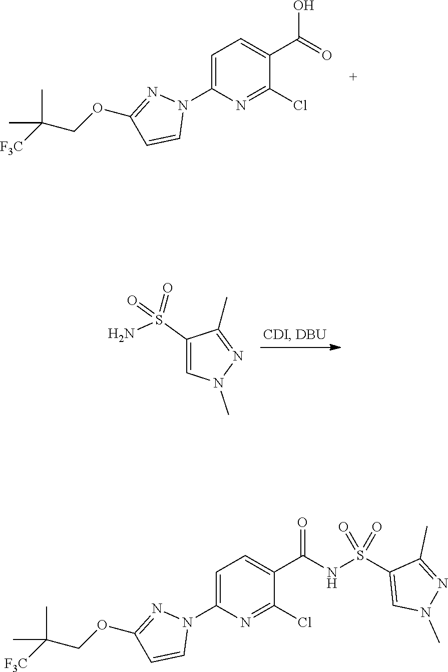 [SMILES: CC(C)(COc1ccn(-c2ccc(C(=O)O)c(Cl)n2)n1)C(F)(F)F ; Cc1nn(C)cc1S(N)(=O)=O ; Cc1nn(C)cc1S(=O)(=O)NC(=O)c1ccc(-n2ccc(OCC(C)(C)C(F)(F)F)n2)nc1Cl]

To a suspension of 2-chloro-6-[3-(3,3,3-trifluoro-2,2-dimethyl-propoxy)pyrazol-1-yl]pyridine-3-carboxylic acid (20.0 g, 53.89 mmol) in THE (78.40 mL) was added solid carbonyldiimidazole (approximately 10.49 g, 64.67 mmol) portion wise and the resulting solution was stirred at room temperature (slight exotherm from 18-21° C. was observed). After 1 h, solid 1,3-dimethylpyrazole-4-sulfonamide (approximately 11.33 g, 64.67 mmol) was added, followed by DBU (approximately 9.845 g, 9.671 mL, 64.67 mmol) in two equal portions over 1 min (exotherm from 19 to 35° C.). The reaction mixture was stirred at room temperature for 16 h. The reaction mixture was diluted with ethyl acetate (118 mL) and then HCl (approximately 107.8 mL of 2 M, 215.6 mmol). The phases were separated and the aqueous phase was extracted with ethyl aceate (78 mL). The combined organics were washed with water (39.2 mL), then brine (40 mL), dried over sodium sulfate and concentrated. The resulting foam was crystallized from a 1:1 isopropanol:heptane mixture (80 mL) to afford 2-chloro-N-((1,3-dimethyl-1H-pyrazol-4-yl)sulfonyl)-6-(3-(3,3,3-trifluoro-2,2-dimethylpropoxy)-1H-pyrazol-1-yl)nicotinamide (26.1 g, 93%) as a white solid. ESI-MS m z calc. 520.0, found 520.9 (M+1)⁺; Retention time: 1.83 minutes.

### Alternative Step G: N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide

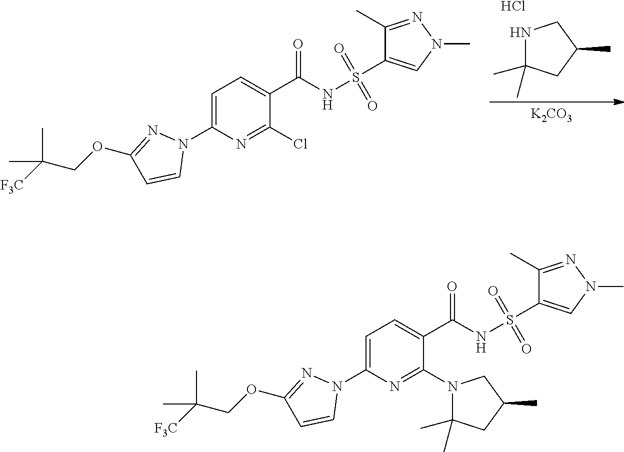

2-chloro-N-(1,3-dimethylpyrazol-4-yl)sulfonyl-6-[3-(3,3,3-trifluoro-2,2-dimethyl-propoxy)pyrazol-1-yl]pyridine-3-carboxamide (20.0 g, 38.39 mmol), (4S)-2,2,4-trimethylpyrrolidine (Hydrochloride salt) (approximately 14.36 g, 95.98 mmol), and K2CO₃ (approximately 26.54 g, 192.0 mmol) were combined in DMSO (80.00 mL) and 1,2-diethoxyethane (20.00 mL) in a 500-mL flask with reflux condenser. The reaction mixture was heated at 120° C. for 16 h then cooled to room temperature. The reaction was diluted with DCM (200.0 mL) and HCl (approximately 172.8 mL of 2 M, 345.5 mmol); aqueous pH ˜1. The phases were separated, and the aqueous phase was extracted with DCM (100.0 mL). The organic phases were combined, washed with water (100.0 mL) (3×), and dried (Na2SO4) to afford an amber solution. The solution was filtered through a DCM-packed silica gel bed (80 g; 4 g/g) and washed with 20% EtOAc/DCM (5×200 mL). The combined filtrate/washes were concentrated to afford 22.2 g of an off-white powder. The powder was slurried in MTBE (140 mL) for 30 min. The solid was collected by filtration (paper/sintered-glass) to afford 24 g after air-drying. The solid was transferred to a drying dish and vacuum-dried (40° C./200 torr/N2 bleed) overnight to afford 20.70 g (90%) of a white powder. ESI-MS m/z calc. 597.2345, found 598.0 (M+1)⁺; Retention time: 2.18 minutes. ¹H NMR (400 MHz, Chloroform-d) δ 13.85 (s, 1H), 8.30 (d, J=8.6 Hz, 1H), 8.23 (d, J=2.8 Hz, 1H), 8.08 (s, 1H), 7.55 (d, J=8.5 Hz, 1H), 5.98 (d, J=2.8 Hz, 1H), 4.24 (s, 2H), 3.86 (s, 3H), 3.44 (dd, J=10.3, 8.4 Hz, 1H), 3.09 (dd, J=10.3, 7.8 Hz, 1H), 2.67-2.52 (m, 1H), 2.47 (s, 3H), 2.12 (dd, J=12.3, 7.8 Hz, 1H), 1.70 (dd, J=12.4, 9.6 Hz, 1H), 1.37 (s, 3H), 1.33 (s, 3H), 1.27 (s, 6H), 1.20 (d, 3H).

### Alternative Synthesis of 3-(3,3,3-trifluoro-2,2-dimethyl-propoxy)-1H-pyrazole

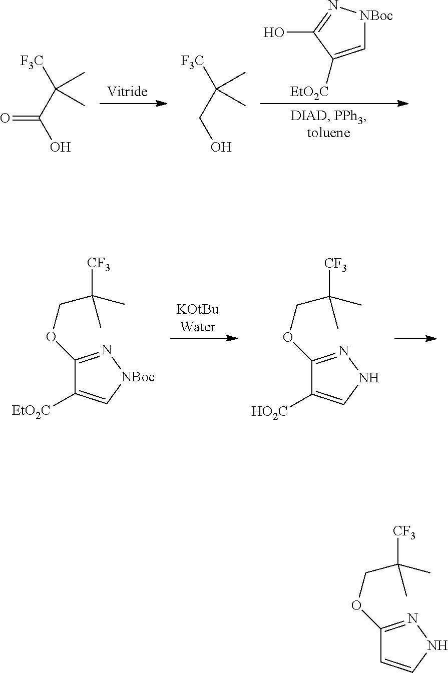

### Step 1: Preparation of 3,3,3-trifluoro-2,2-dimethylpropan-1-oI

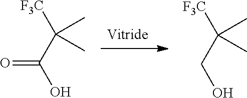

A reactor was loaded with toluene (300 mL) and 3,3,3-trifluoro-2,2-dimethylpropanoic acid (30 g, 192.2 mmol), capped, purged under nitrogen. The reaction was set to control the internal temperature to 40° C. A solution of Vitride (65% in toluene, approximately 119.6 g of 65% w/w, 115.4 mL of 65% w/w, 384.4 mmol) was set up for addition via syringe, and addition was begun at 40° C., with the target addition temperature between 4° and 50° C. The reaction was stirred at 40° C. for 90 min. The reaction was cooled to 10° C. then the remaining Vitride was quenched with slow addition of water (6 mL). A solution of 15% aq NaOH (30 mL) was added in portions, and solids precipitated half way through the base addition. Water (60.00 mL) was added. The mixture was warmed to 30° C. and held for at least 15 mins. The mixture was then cooled to 20° C. The aqueous layer was removed. The organic layer was washed with water (60 mL×3), and then washed with brine (60 mL). The washed organic layer was dried under Na₂SO₄, followed with MgSO₄. The mix was filtered through Celite, and the cake washed with toluene (60.00 mL) and pulled dry. The product 3,3,3-trifluoro-2,2-dimethyl-propan-1-ol (22.5 g, 82%) was obtained as clear colorless solution.

### Step 2: Preparation of 1-(tert-butyl) 4-ethyl 3-(3,3,3-trifluoro-2,2-dimethylpropoxy)-1H-pyrazole-1,4-dicarboxylate

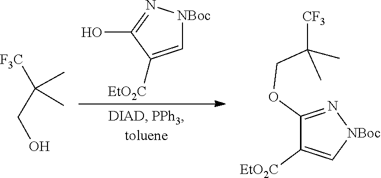

A reactor was charged with 3,3,3,-trifluoro-2,2-dimethylpropan-1-ol (17.48 g, 123.0 mmol) solution in toluene (250g), 1-(tert-butyl) 4-ethyl 3-hydroxy-1H-pyrazole-1,4-dicarboxylate (30.0 g, 117.1 mmol), and PPh₃ (35.33 g, 134.7 mmol). The reaction was heated to 40° C. DIAD (26.09 mL, 134.7 mmol) was weighed and placed into a syringe and added over 10 minutes while maintaining an internal temperature ranging between 4° and 50° C. The reaction was then heated to 100° C. over 30 minutes. After holding at 100° C. for 30 minutes, the reaction was complete, and the mixture was cooled to 70° C. over 15 minutes. Heptane (180.0 mL) was added, and the jacket was cooled to 15° C. over 1 hour. (TPPO began crystallizing at ˜35° C.). The mixture stirring at 15° C. was filtered (fast), the cake was washed with a pre-mixed solution of toluene (60 mL) and heptane (60 mL) and then pulled dry. The clear solution was concentrated to a waxy solid (45° C., vacuum, rotovap). Crude 1-(tert-butyl) 4-ethyl 3-(3,3,3-trifluoro-2,2-dimethylpropoxy)-1H-pyrazole-1,4-dicarboxylate (53.49g) was obtained as a waxy solid, (˜120% of theoretical mass recovered).

### Step 3: Preparation of 3-(3,3,3-trifluoro-2,2-dimethylpropoxy)-1H-pyrazole-4-carboxylic acid

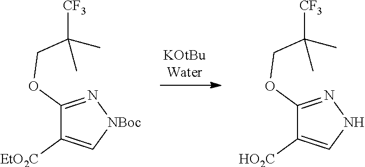 [SMILES: CCOC(=O)c1cn(C(=O)OC(C)(C)C)nc1OCC(C)(C)C(F)(F)F ; CC(C)(COc1n[nH]cc1C(=O)O)C(F)(F)F]

A solution of 1-(tert-butyl) 4-ethyl 3-(3,3,3-trifluoro-2,2-dimethylpropoxy)-1H-pyrazole-1,4-dicarboxylate (50.0 g, 131 mmol) in 2-methyltetrahydrofuran (500 mL) was prepared in a reactor and stirred at 40° C. Portions of KOt-Bu (80.85 g, 720.5 mmol) were then added over 30 minutes. Addition was exothermic. After 20 53.49g UPLC-MS showed complete removal of the Boc group, so water (3.53 g, 3.53 mL, 196 mmol) was added drop-wise addition via syringe over 20 min to keep the reaction temperature between 40-50° C. The mixture was then stirred for 17 hours to complete the reaction. The mixture was then cooled to 20° C. and water (400 mL) was added. The stirring was stopped and the layers were separated. The desired product in the aqueous layer was returned to the reactor and the organic layer was discarded. The aqueous layer was washed with 2-Me-THF (200 mL). Isopropanol (50. mL) was added followed by dropwise addition of aqueous HCl (131 mL of 6.0 M, 786.0 mmol) to adjust the pH to <3 while maintaining the temperature below 30° C. The resulting solid was then isolated by filtration and the filter cake washer with water (100 mL) then pulled dry until a sticky cake was obtained. The solids were then dried under vacuum at 55° C. to afford 3-(3,3,3-trifluoro-2,2-dimethylpropoxy)-1H-pyrazole-4-carboxylic acid (23.25 g) as an off-white fine solid.

### Step 4: Preparation of 3-(3,3,3-trifluoro-2,2-dimethyl-propoxy)-1H-pyrazole

3-(3,3,3-trifluoro-2,2-dimethylpropoxy)-1H-pyrazole-4-carboxylic acid (1.0 equiv) was added to a reactor followed by DMF (6.0 vol, 2.6 wt equiv). The mixture was stirred at 18-22° C. DBU (0.2 equiv.) was charged to the reaction mixture at a rate of approximately 45 mL/min. The reaction temperature was then raised to 98-102° C. over 45 minutes. The reaction mixture was stirred at 98-102° C. for no less than 10 h. The reaction mixture was then cooled to −2° C. to 2° C. over approximately 1 hour and was used without isolation to make ethyl 2-chloro-6-(3-(3,3,3-trifluoro-2,2-dimethylpropoxy)-1H-pyrazol-1-yl)nicotinate.

### Alternate procedure for the preparation of 2-chloro-6-[3-(3,3,3-trifluoro-2,2-dimethyl-propoxy)pyrazol-1-yl]pyridine-3-carboxylic acid

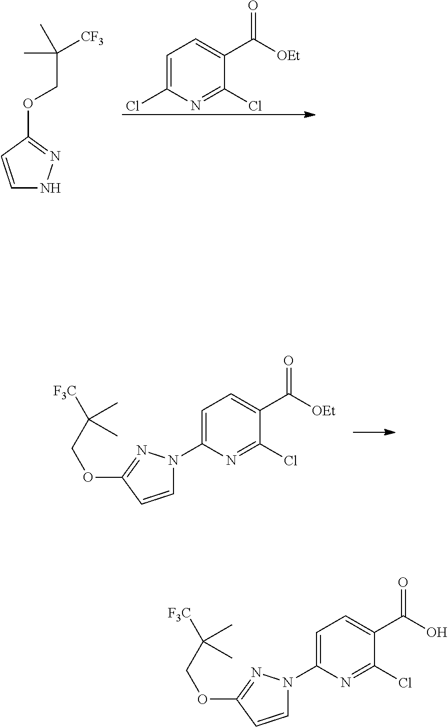 [SMILES: CC(C)(COc1cc[nH]n1)C(F)(F)F ; CCOC(=O)c1ccc(Cl)nc1Cl ; CCOC(=O)c1ccc(-n2ccc(OCC(C)(C)C(F)(F)F)n2)nc1Cl ; CC(C)(COc1ccn(-c2ccc(C(=O)O)c(Cl)n2)n1)C(F)(F)F]

### Step 1. Ethyl 2-chloro-6-(3-(3,3,3-trifluoro-2,2-dimethylpropoxy)-1H-pyrazol-1-yl)nicotinate

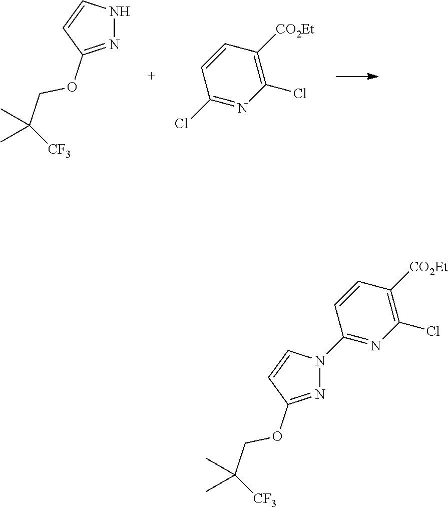 [SMILES: CC(C)(COc1cc[nH]n1)C(F)(F)F ; CCOC(=O)c1ccc(Cl)nc1Cl ; CCOC(=O)c1ccc(-n2ccc(OCC(C)(C)C(F)(F)F)n2)nc1Cl]

A solution of ethyl 2,6-dichloronicotinate (256 g, 1.16 mol) and 3-(3,3,3-trifluoro-2,2-dimethyl-propoxy)-1H-pyrazole (242 g, 1.16 mol) in DMF (1.53 L) was treated with potassium carbonate (209 g, 1.51 mol) and DABCO (19.6 g, 174 mmol). The resultant suspension was stirred allowed to exotherm from 14 to 25° C. and then maintained at 20-25° C. with external cooling for 3 days. The suspension was cooled to below 10° C. when water (2.0 L) was added in a thin stream while maintaining the temperature below 25° C. After the addition was complete, the suspension was stirred for an additional 1 h. The solid was collected by filtration (sintered-glass/polypad) and the filter-cake was washed with water (2×500-mL) and dried with suction for 2 h to afford water-damp ethyl 2-chloro-6-(3-(3,3,3-trifluoro-2,2-dimethylpropoxy)-1H-pyrazol-1-yl)nicotinate (512 g; 113% yield) as white powder which was used without further steps in the subsequent reaction.

### Step 2. 2-chloro-6-(3-(3,3,3-trifluoro-2,2-dimethylpropoxy)-1h-pyrazol-1-yl)nicotinic acid

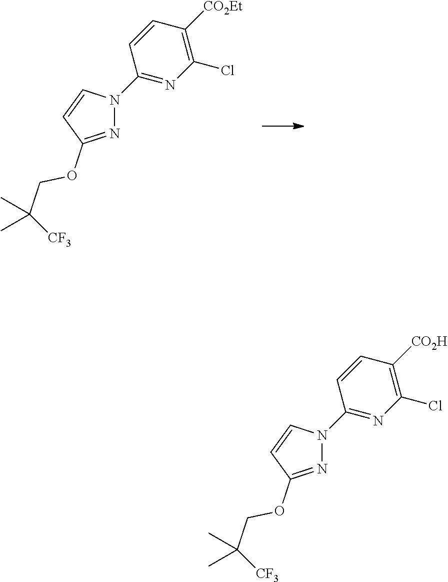 [SMILES: CCOC(=O)c1ccc(-n2ccc(OCC(C)(C)C(F)(F)F)n2)nc1Cl ; CC(C)(COc1ccn(-c2ccc(C(=O)O)c(Cl)n2)n1)C(F)(F)F]

The water-damp ethyl 2-chloro-6-(3-(3,3,3-trifluoro-2,2-dimethylpropoxy)-1H-pyrazol-1-yl)nicotinate (455 g, 1.16 mol; assumed 100% yield from previous step) in EtOH (1.14 L) and THE (455 mL) was stirred at ambient temperature (17° C.) when 1 M NaOH (1.16 L, 1.16 mol) was added. The reaction mixture exothermed to 30° C. and was further warmed at 40° C. for 2 h. The solution was quenched with 1 M HCl (1.39 L, 1.39 mol) which resulted in an immediate precipitation which became thicker as the acid was added. The creamy suspension was allowed to cool to room temperature and was stirred overnight. The solid was collected by filtration (sintered-glass/poly pad). The filter-cake was washed with water (2×500-mL). The filter-cake was dried by suction for 1 h but remained wet. The damp solid was transferred to a 10-L Buchi flask for further drying (50° C./20 torr), but was not effective. Further effort to dry by chasing with i-PrOH was also ineffective. Successful drying was accomplished after the damp solid was backfilled with i-PrOAc (3 L), the suspension was heated at 60° C. (homogenization), and re-concentrated to dryness (50° C./20 torr) to afford dry 2-chloro-6-(3-(3,3,3-trifluoro-2,2-dimethylpropoxy)-1h-pyrazol-1-yl)nicotinic acid (408 g; 97% yield for two steps) as a fine, white powder. The product was further dried in a vacuum oven (50° C./10 torr/N2 bleed) for 2 h but marginal weight loss was observed. 1H NMR (400 MHz, DMSO-d6) δ 13.64 (s, 1H), 8.49-8.36 (m, 2H), 7.77 (d, J=8.4 Hz, 1H), 6.26 (d, J=2.8 Hz, 1H), 4.28 (s, 2H), 1.24 (s, 6H). 19F NMR (376 MHz, DMSO-d6) δ -75.2. KF analysis: 0.04% water.

### Preparation of Form A of Compound I

The crystalline Form A of Compound I was obtained as a result of the following synthesis. Combined 2-chloro-N-(1,3-dimethylpyrazol-4-yl)sulfonyl-6-[3-(3,3,3-trifluoro-2,2-dimethyl-propoxy)pyrazol-1-yl]pyridine-3-carboxamide(108 g, 207.3 mmol), (4S)-2,2,4-trimethylpyrrolidine (Hydrochloride salt) (77.55 g, 518.2 mmol), was combined with K₂CO₃(143.2 g, 1.036 mol) in DMSO (432.0 mL) and 1,2-diethoxyethane (108.0 mL) in a 1-L RB flask with a reflux condenser. The resulting suspension was heated at 120° C. and was stirred at temperature overnight. Then the reaction was diluted with DCM (1.080 L) and HCl (933.0 mL of 2 M, 1.866 mol) was slowly added. The liquid phases were separated, and the aqueous phase was extracted with DCM (540.0 mL). The organic phases were combined, washed with water (540.0 mL) (3×), then dried with (Na₂SO₄) to afford an amber solution. Silica gel (25 g) was added and then the drying agent/silica gel was filtered off. The filter-bed was washed with DCM (3×50-mL). The organic phases were combined and concentrated (40° C./40 torr) to afford crude N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (198.6 g, 160% theory) as an off-white solid. The solid was diluted with MTBE (750 mL), warmed at 60° C. (external temperature), and mixed to a homogenous suspension. The suspension was cooled to 30° C. with stirring and the solid was collected by filtration, air-dried, and vacuum-dried to afford Compound I (111.1 g; 90%) as a fine, white powder.

The crystalline Form A of Compound I was also obtained through the following procedure. A suspension of Compound I (150.0 g, 228.1 mmol) in iPrOH (480 mL) and water (120 mL) was heated at 82° C. to obtain a solution. The solution was cooled with a J-Kem controller at a cooling rate of 10° C./h. Once the temperature reached 74° C., the solution was seeded with a sample of Compound I in crystalline Form A. Crystallization occurred immediately. The sample was cooled to −5° C., let stir for 1 h, and then the solid was collected by filtration (sintered glass/paper). The filter-cake was washed with i-PrOH (75 mL) (2×), air-dried with suction, air-dried in a drying dish (120.6 g mostly dried), vacuum-dried (55° C./300 torr/N2 bleed) for 4 h, and then RT overnight. Overnight drying afforded 118.3 g (87% yield) of a white powder.

A suspension of Compound I (116 g, 176.3 mmol) in iPrOH (371 mL) and water (93 mL) was heated at 82° C. to obtain a solution. The solution was cooled to 20° C. with a J-Kem controller at a cooling rate of 10° C./h. Once the temperature reached 74° C., the solution was seeded with a sample of Compound I in crystalline Form A. Crystallization occurred immediately. Cooling was stopped at 20° C. and the mixture was stirred overnight. The solid was collected by filtration, washed with i-PrOH (2×75 mL), air-dried with suction, and vacuum-dried (55° C./300 torr/N2 bleed) to afford Compound I Form A (103.3 g) as a white powder.

### Example 2: Synthesis of (R)-1-(2,2-Difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II)

The synthesis of Compound II is described in United States Patent Publication US 2009/0131492, incorporated herein by reference for this disclosure. The production of solid dispersions of Compound II is described in International Patent Publications WO 2011/119984 and WO 2015/160787, the contents of which are hereby incorporated by reference in their entirety.

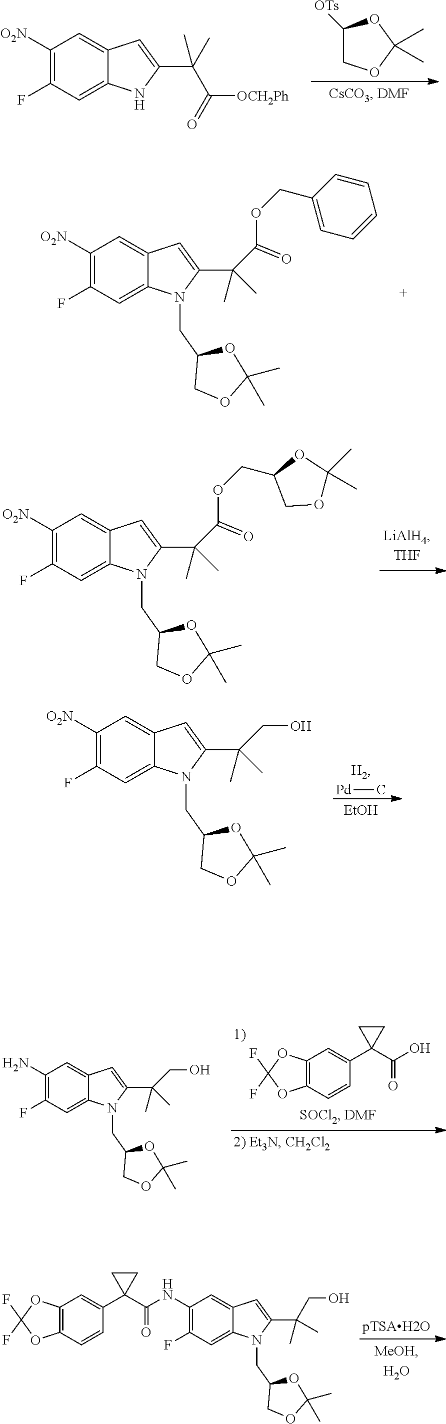

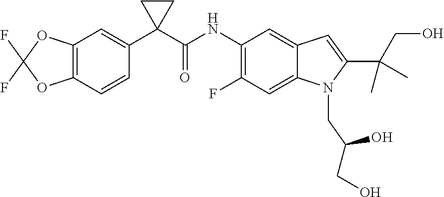 [SMILES: CC(C)(CO)c1cc2cc(NC(=O)C3(c4ccc5c(c4)OC(F)(F)O5)CC3)c(F)cc2n1C[C@@H](O)CO]

### Step 1: (R)-Benzyl 2-(1-((2,2-dimethyl-1,3-dioxolan-4-yl)methyl)-6-fluoro-5-nitro-1H-indol-2-yl)-2-methylpropanoate and ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate

Cesium carbonate (8.23 g, 25.3 mmol) was added to a mixture of benzyl 2-(6-fluoro-5-nitro-1H-indol-2-yl)-2-methylpropanoate (3.0 g, 8.4 mmol) and (S)-(2,2-dimethyl-1,3-dioxolan-4-yl)methyl 4-methylbenzenesulfonate (7.23 g, 25.3 mmol) in DMF (N,N-dimethylformamide) (17 mL). The reaction was stirred at 80° C. for 46 hours under a nitrogen atmosphere. The mixture was then partitioned between ethyl acetate and water. The aqueous layer was extracted with ethyl acetate. The combined ethyl acetate layers were washed with brine, dried over MgSO₄, filtered and concentrated. The crude product, a viscous brown oil which contains both of the products shown above, was taken directly to the next step without further purification. (R)-Benzyl 2-(1-((2,2-dimethyl-1,3-dioxolan-4-yl)methyl)-6-fluoro-5-nitro-1H-indol-2-yl)-2-methylpropanoate, ESI-MS m z calc. 470.2, found 471.5 (M+1)*. Retention time 2.20 minutes. ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate, ESI-MS m z calc. 494.5, found 495.7 (M+1)*. Retention time 2.01 minutes.

### Step 2: (R)-2-(1-((2,2-dimethyl-1,3-dioxolan-4-yl)methyl)-6-fluoro-5-nitro-1H-indol-2-yl)-2-methylpropan-1-ol

The crude reaction mixture obtained in step (A) was dissolved in THE (tetrahydrofuran) (42 mL) and cooled in an ice-water bath. LiAlH₄ (16.8 mL of 1 M solution, 16.8 mmol) was added drop-wise. After the addition was complete, the mixture was stirred for an additional 5 minutes. The reaction was quenched by adding water (1 mL), 15% NaOH solution (1 mL) and then water (3 mL). The mixture was filtered over Celite, and the solids were washed with THF and ethyl acetate. The filtrate was concentrated and purified by column chromatography (30-60% ethyl acetate-hexanes) to obtain (R)-2-(1-((2,2-dimethyl-1,3-dioxolan-4-yl)methyl)-6-fluoro-5-nitro-1H-indol-2-yl)-2-methylpropan-1-ol as a brown oil (2.68 g, 87% over 2 steps). ESI-MS m z calc. 366.4, found 367.3 (M+1)*. Retention time 1.68 minutes. ¹H NMR (400 MHz, DMSO-d6) δ 8.34 (d, J=7.6 Hz, 1H), 7.65 (d, J=13.4 Hz, 1H), 6.57 (s, 1H), 4.94 (t, J=5.4 Hz, 1H), 4.64-4.60 (m, 1H), 4.52-4.42 (m, 2H), 4.16-4.14 (m, 1H), 3.76-3.74 (m, 1H), 3.63-3.53 (m, 2H), 1.42 (s, 3H), 1.38-1.36 (m, 6H) and 1.19 (s, 3H) ppm. (DMSO is dimethylsulfoxide).

### Step 3: (R)-2-(5-amino-1-((2,2-dimethyl-1,3-dioxolan-4-yl)methyl)-6-fluoro-1H-indol-2-yl)-2-methylpropan-1-ol

(R)-2-(1-((2,2-dimethyl-1,3-dioxolan-4-yl)methyl)-6-fluoro-5-nitro-1H-indol-2-yl)-2-methylpropan-1-ol (2.5 g, 6.82 mmol) was dissolved in ethanol (70 mL) and the reaction was flushed with N₂. Then Pd-C(250 mg, 5% wt) was added. The reaction was flushed with nitrogen again and then stirred under H₂ (atm). After 2.5 hours only partial conversion to the product was observed by LCMS. The reaction was filtered through Celite and concentrated. The residue was re-subjected to the conditions above. After 2 hours LCMS indicated complete conversion to product. The reaction mixture was filtered through Celite. The filtrate was concentrated to yield the product (1.82 g, 79%). ESI-MS m z calc. 336.2, found 337.5 (M+1)*. Retention time 0.86 minutes. ¹H NMR (400 MHz, DMSO-d6) δ 7.17 (d, J=12.6 Hz, 1H), 6.76 (d, J=9.0 Hz, 1H), 6.03 (s, 1H), 4.79-4.76 (m, 1H), 4.46 (s, 2H), 4.37-4.31 (m, 3H), 4.06 (dd, J=6.1, 8.3 Hz, 1H), 3.70-3.67 (m, 1H), 3.55-3.52 (m, 2H), 1.41 (s, 3H), 1.32 (s, 6H) and 1.21 (s, 3H) ppm.

### Step 4: (R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide

DMF (3 drops) was added to a stirring mixture of 1-(2,2-difluorobenzo[d][1,3]dioxol-5-yl)cyclopropanecarboxylic acid (1.87 g, 7.7 mmol) and thionyl chloride (1.30 mL, 17.9 mmol). After 1 hour a clear solution had formed. The solution was concentrated under vacuum and then toluene (3 mL) was added and the mixture was concentrated again. The toluene step was repeated once more and the residue was placed on high vacuum for 10 minutes. The acid chloride was then dissolved in dichloromethane (10 mL) and added to a mixture of (R)-2-(5-amino-1-((2,2-dimethyl-1,3-dioxolan-4-yl)methyl)-6-fluoro-1H-indol-2-yl)-2-methylpropan-1-ol (1.8 g, 5.4 mmol) and triethylamine (2.24 mL, 16.1 mmol) in dichloromethane (45 mL). The reaction was stirred at room temperature for 1 hour. The reaction was washed with 1N HCl solution, saturated NaHCO₃ solution and brine, dried over MgSO₄ and concentrated to yield the product (3 g, 100%). ESI-MS m z calc. 560.6, found 561.7 (M+1)+. Retention time 2.05 minutes. ¹H NMR (400 MHz, DMSO-d6) δ 8.31 (s, 1H), 7.53 (s, 1H), 7.42-7.40 (m, 2H), 7.34-7.30 (m, 3H), 6.24 (s, 1H), 4.51-4.48 (m, 1H), 4.39-4.34 (m, 2H), 4.08 (dd, J=6.0, 8.3 Hz, 1H), 3.69 (t, J=7.6 Hz, 1H), 3.58-3.51 (m, 2H), 1.48-1.45 (m, 2H), 1.39 (s, 3H), 1.34-1.33 (m, 6H), 1.18 (s, 3H) and 1.14-1.12 (m, 2H) ppm.

### Step 5: (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II)

(R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropane carboxamide (3.0 g, 5.4 mmol) was dissolved in methanol (52 mL). Water (5.2 mL) was added followed by p-TsOH·H₂O (p-toluenesulfonic acid hydrate) (204 mg, 1.1 mmol). The reaction was heated at 80° C. for 45 minutes. The solution was concentrated and then partitioned between ethyl acetate and saturated NaHCO₃ solution. The ethyl acetate layer was dried over MgSO₄ and concentrated. The residue was purified by column chromatography (50-100% ethyl acetate—hexanes) to yield the product. (1.3 g, 47%, ee >98% by SFC). ESI-MS m z calc. 520.5, found 521.7 (M+1)+. Retention time 1.69 minutes. ¹H NMR (400 MHz, DMSO-d6) δ 8.31 (s, 1H), 7.53 (s, 1H), 7.42-7.38 (m, 2H), 7.33-7.30 (m, 2H), 6.22 (s, 1H), 5.01 (d, J=5.2 Hz, 1H), 4.90 (t, J=5.5 Hz, 1H), 4.75 (t, J=5.8 Hz, 1H), 4.40 (dd, J=2.6, 15.1 Hz, 1H), 4.10 (dd, J=8.7, 15.1 Hz, 1H), 3.90 (s, 1H), 3.65-3.54 (m, 2H), 3.48-3.33 (m, 2H), 1.48-1.45 (m, 2H), 1.35 (s, 3H), 1.32 (s, 3H) and 1.14-1.11 (m, 2H) ppm.

### Preparation of a Solid Dispersion of Amorphous Compound II

A solvent system of dichloromethane (DCM) and methanol (MeOH), is formulated according to the ratio 80 wt % DCM/20 wt % MeOH, in an appropriately sized container, equipped with a magnetic stirrer and stir plate. Into this solvent system, hypromellose polymer (HPMC, E15 grade) and Compound II were added according to the ratio 20 wt % hypromellose/80 wt % Compound II. The resulting mixture contained 12.5 wt % solids. The actual amounts of ingredients and solvents used to generate this mixture are recited in the table below:

### Solid spray dispersion ingredients for amorphous Compound II

| | Units | Batch |
|---|---|---|
| Compound II | g | 2400 |
| HPMC | g | 600 |
| Total Solids | g | 3000 |
| DCM | g | 16800 |
| MeOH | g | 4200 |
| Total Solvents | g | 21000 |
| Total Spray Solution Weight | g | 24000 |

The mixture was mixed until it was substantially homogenous and all components were substantially dissolved.

A spray drier, Anhydro MS-35 Spray Drier, fitted with two fluid 0.8 mm nozzle (Schlick series 970/0 S4), was used under normal spray drying mode, following the dry spray process parameters recited in the table below.

### Processing, parameters to generate solid spray dispersion of amorphous Compound 11

| Parameter: | Value: | |
|---|---|---|
| Process Gas Flow Rate | 34 | Kg/hr |
| Nozzle Gas Flow Rate | 4.2 | Kg/hr |
| Feed Flow Rate | 2 | Kg/hr |
| Inlet Temperature | 96-108° | C. |
| Outlet Temperature | 40° | C. |
| Vacuum Dryer Temperature | 45° | C. |
| Vacuum Drying Time | 24-72 | hours |

A high efficiency cyclone separated the wet product from the spray gas and solvent vapors. The wet product was transferred into trays and placed in vacuum dryer for drying to reduce residual solvents to a level of less than about 3000 ppm for MeOH and less than 600 ppm of DCM and to generate dry dispersion of amorphous Compound II, containing <0.02%0 MeOH and <0.0600 DCM.

### Example 3: Synthesis of N-(2,4-di-tert-butyl-5-hydroxyphenyl)-4-oxo-1,4-dihydroquinoline-3-carboxamide (Compound III)

The synthesis of Compound III is described in International Patent Publication WO 2006/002421, incorporated herein by reference for this disclosure. The production of solid dispersions of Compound III is described in International Patent Publications WO 2007/079139 and WO 2013/130669, the contents of which are hereby incorporated by reference in their entirety.

### Part A: Synthesis of 4-oxo-1,4-dihydroquinoline-3-carboxylic acid

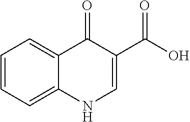 [SMILES: O=C(O)c1c[nH]c2ccccc2c1=O]

### Step 1: 2-Phenylaminomethylene-malonic acid diethyl ester

A mixture of aniline (25.6 g, 0.275 mol) and diethyl 2-(ethoxymethylene)malonate (62.4 g, 0.288 mol) was heated at 140-150° C. for 2 h. The mixture was cooled to room temperature and dried under reduced pressure to afford 2-phenylaminomethylene-malonic acid diethyl ester as a solid, which was used in the next step without further purification. ¹H NMR (DMSO-d₆) δ 11.00 (d, 1H), 8.54 (d, J=13.6 Hz, 1H), 7.36-7.39 (m, 2H), 7.13-7.17 (m, 3H), 4.17-4.33 (m, 4H), 1.18-1.40 (m, 6H).

### Step 2: 4-Hydroxyquinoline-3-carboxylic acid ethyl ester

A 1 L three-necked flask fitted with a mechanical stirrer was charged with 2-phenylaminomethylene-malonic acid diethyl ester (26.3 g, 0.100 mol), polyphosphoric acid (270 g) and phosphoryl chloride (750 g). The mixture was heated to 70° C. and stirred for 4 h. The mixture was cooled to room temperature and filtered. The residue was treated with aqueous Na₂CO₃ solution, filtered, washed with water and dried. 4-Hydroxyquinoline-3-carboxylic acid ethyl ester was obtained as a pale brown solid (15.2 g, 70%). The crude product was used in next step without further purification.

### Step 3: 4-Oxo-1,4-dihydroquinoline-3-carboxylic acid

4-Hydroxyquinoline-3-carboxylic acid ethyl ester (15 g, 69 mmol) was suspended in sodium hydroxide solution (2N, 150 mL) and stirred for 2 h at reflux. After cooling, the mixture was filtered, and the filtrate was acidified to pH 4 with 2N HCl. The resulting precipitate was collected via filtration, washed with water and dried under vacuum to give 4-oxo-1,4-dihydroquinoline-3-carboxylic acid as a pale white solid (10.5 g, 92%). ¹H NMR (DMSO-d₆) δ 15.34 (s, 1H), 13.42 (s, 1H), 8.89 (s, 1H), 8.28 (d, J=8.0 Hz, 1H), 7.88 (m, 1H), 7.81 (d, J=8.4 Hz, 1H), 7.60 (m, 1H).

### Part B: Synthesis of N-(2,4-di-tert-butyl-5-hydroxyphenyl)-4-oxo-1,4-dihydroquinoline-3-carboxamide

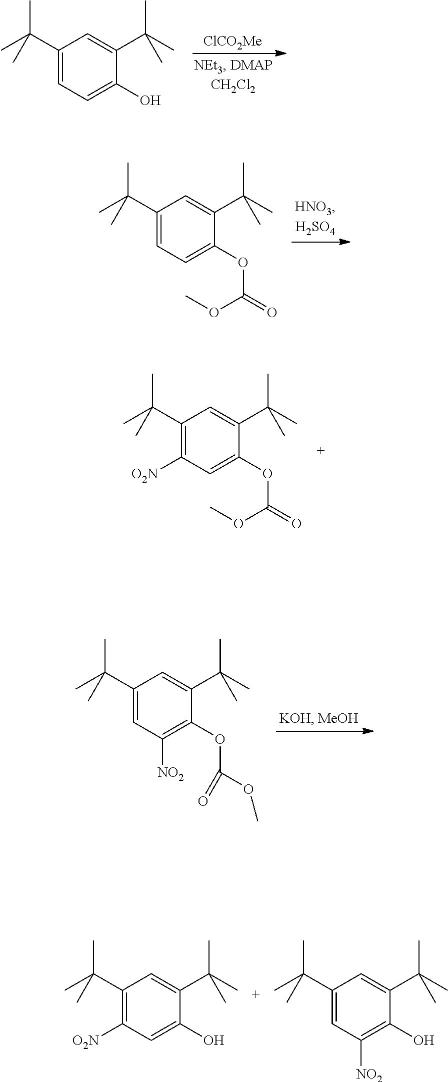

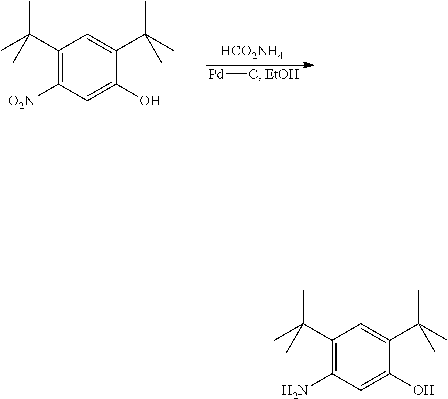

### Step 1: Carbonic acid 2,4-di-tert-butyl-phenyl ester methyl ester

Methyl chloroformate (58 mL, 750 mmol) was added dropwise to a solution of 2,4-di-tert-butyl-phenol (103.2 g, 500 mmol), Et₃N (139 mL, 1000 mmol) and DMAP (3.05 g, 25 mmol) in dichloromethane (400 mL) cooled in an ice-water bath to 0° C. The mixture was allowed to warm to room temperature while stirring overnight, then filtered through silica gel (approx. 1L) using 10% ethyl acetate—hexanes (˜4 L) as the eluent. The combined filtrates were concentrated to yield carbonic acid 2,4-di-tert-butyl-phenyl ester methyl ester as a yellow oil (132 g, quant.). ¹H NMR (400 MHz, DMSO-d₆) δ 7.35 (d, J=2.4 Hz, 1H), 7.29 (dd, J=8.5, 2.4 Hz, 1H), 7.06 (d, J=8.4 Hz, 1H), 3.85 (s, 3H), 1.30 (s, 9H), 1.29 (s, 9H).

### Step 2: Carbonic acid 2,4-di-tert-butyl-5-nitro-phenyl ester methyl ester and Carbonic acid 2,4-di-tert-butyl-6-nitro-phenyl ester methyl ester

To a stirring mixture of carbonic acid 2,4-di-tert-butyl-phenyl ester methyl ester (4.76 g, 180 mmol) in conc. sulfuric acid (2 mL), cooled in an ice-water bath, was added a cooled mixture of sulfuric acid (2 mL) and nitric acid (2 mL). The addition was done slowly so that the reaction temperature did not exceed 50° C. The reaction was allowed to stir for 2 h while warming to room temperature. The reaction mixture was then added to ice-water and extracted into diethyl ether. The ether layer was dried (MgSO₄), concentrated and purified by column chromatography (0-10% ethyl acetate—hexanes) to yield a mixture of carbonic acid 2,4-di-tert-butyl-5-nitro-phenyl ester methyl ester and carbonic acid 2,4-di-tert-butyl-6-nitro-phenyl ester methyl ester as a pale yellow solid (4.28 g), which was used directly in the next step.

### Step 3: 2,4-Di-tert-butyl-5-nitro-phenol and 2,4-Di-tert-butyl-6-nitro-phenol

The mixture of carbonic acid 2,4-di-tert-butyl-5-nitro-phenyl ester methyl ester and carbonic acid 2,4-di-tert-butyl-6-nitro-phenyl ester methyl ester (4.2 g, 14.0 mmol) was dissolved in MeOH (65 mL) before KOH (2.0 g, 36 mmol) was added. The mixture was stirred at room temperature for 2 h. The reaction mixture was then made acidic (pH 2-3) by adding conc. HCl and partitioned between water and diethyl ether. The ether layer was dried (MgSO₄), concentrated and purified by column chromatography (0-5% ethyl acetate-hexanes) to provide 2,4-di-tert-butyl-5-nitro-phenol (1.31 g, 29% over 2 steps) and 2,4-di-tert-butyl-6-nitro-phenol. 2,4-Di-tert-butyl-5-nitro-phenol: ¹H NMR (400 MHz, DMSO-d₆) δ 10.14 (s, 1H, OH), 7.34 (s, 1H), 6.83 (s, 1H), 1.36 (s, 9H), 1.30 (s, 9H). 2,4-Di-tert-butyl-6-nitro-phenol ¹H NMR (400 MHz, CDCl₃) δ 11.48 (s, 1H), 7.98 (d, J=2.5 Hz, 1H), 7.66 (d, J=2.4 Hz, 1H), 1.47 (s, 9H), 1.34 (s, 9H).

### Step 4: 5-Amino-2,4-di-tert-butyl-phenol

To a refluxing solution of 2,4-di-tert-butyl-5-nitro-phenol (1.86 g, 7.40 mmol) and ammonium formate (1.86 g) in ethanol (75 mL) was added Pd-5% wt. on activated carbon (900 mg). The reaction mixture was stirred at reflux for 2 h, cooled to room temperature and filtered through Celite. The Celite was washed with methanol and the combined filtrates were concentrated to yield 5-amino-2,4-di-tert-butyl-phenol as a grey solid (1.66 g, quant.). ¹H NMR (400 MHz, DMSO-d₆) δ 8.64 (s, 1H, OH), 6.84 (s, 1H), 6.08 (s, 1H), 4.39 (s, 2H, NH₂), 1.27 (m, 18H); HPLC ret. time 2.72 min, 10-99% CH₃CN, 5 min run; ESI-MS 222.4 m/z [M+H]⁺.

### Step 5: N-(5-hydroxy-2,4-di-tert-butyl-phenyl)-4-oxo-1H-quinoline-3-carboxamide

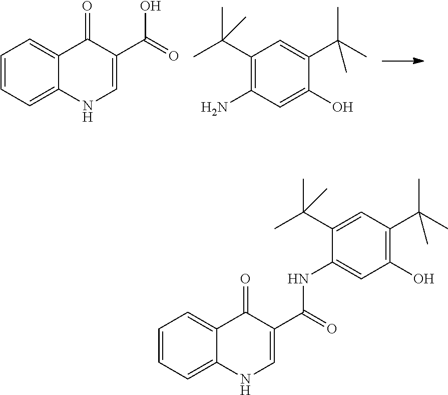

To a suspension of 4-oxo-1,4-dihydroquinolin-3-carboxylic acid (35.5 g, 188 mmol) and HBTU (85.7 g, 226 mmol) in DMF (280 mL) was added Et₃N (63.0 mL, 451 mmol) at ambient temperature. The mixture became homogeneous and was allowed to stir for 10 min before 5-amino-2,4-di-tert-butyl-phenol (50.0 g, 226 mmol) was added in small portions. The mixture was allowed to stir overnight at ambient temperature. The mixture became heterogeneous over the course of the reaction. After all of the acid was consumed (LC-MS analysis, MH+190, 1.71 min), the solvent was removed in vacuo. EtOH (ethyl alcohol) was added to the orange solid material to produce a slurry. The mixture was stirred on a rotovap (bath temperature 65° C.) for 15 min without placing the system under vacuum. The mixture was filtered and the captured solid was washed with hexanes to provide a white solid that was the EtOH crystalate. Et₂O (diethyl ether) was added to the solid obtained above until a slurry was formed. The mixture was stirred on a rotovapor (bath temperature 25° C.) for 15 min without placing the system under vacuum. The mixture was filtered and the solid captured. This procedure was performed a total of five times. The solid obtained after the fifth precipitation was placed under vacuum overnight to provide N-(5-hydroxy-2,4-di-tert-butyl-phenyl)-4-oxo-1H-quinoline-3-carboxamide (38 g, 52%). HPLC ret. time 3.45 min, 10-99% CH₃CN, 5 min run; ¹H NMR (400 MHz, DMSO-d₆) δ 12.88 (s, 1H), 11.83 (s, 1H), 9.20 (s, 1H), 8.87 (s, 1H), 8.33 (dd, J=8.2, 1.0 Hz, 1H), 7.83-7.79 (m, 1H), 7.76 (d, J=7.7 Hz, 1H), 7.54-7.50 (m, 1H), 7.17 (s, 1H), 7.10 (s, 1H), 1.38 (s, 9H), 1.37 (s, 9H); ESI-MS m/z calc'd 392.21; found 393.3 [M+H]⁺.

### Solid Dispersion of Amorphous Compound III

A solvent system of MEK and DI water, formulated according to the ratio 90 wt % MEK/10 wt % DI water, was heated to a temperature of 20-30° C. in a reactor, equipped with a magnetic stirrer and thermal circuit. Into this solvent system, hypromellose acetate succinate polymer (HPMCAS)(HG grade), SLS, and N-[2,4-Bis(1,1-dimethylethyl)-5-hydroxyphenyl]-1,4-dihydro-4-oxoquinoline-3-carboxamide were added according to the ratio 19.5 wt % o hypromellose acetate succinate/0.5 wt % o SLS/80 wt % o N-[2,4-Bis(1,1-dimethylethyl)-5-hydroxyphenyl]-1,4-dihydro-4-oxoquinoline-3-carboxamide. The resulting mixture contained 10.5 wt % o solids. The actual amounts of ingredients and solvents used to generate this mixture are recited in the table below:

### Spray Dispersion Ingredients for Compound III

| | Units | Batch |
|---|---|---|
| N-[2,4-Bis(1,1-dimethylethyl)-5-hydroxyphenyl]-1,4- | Kg | 70.0 |
| dihydro-4-oxoquinoline-3-carboxamide | | |
| HPMCAS | Kg | 17.1 |
| SLS | Kg | 0.438 |
| Total Solids | Kg | 87.5 |
| MEK | Kg | 671 |
| Water | Kg | 74.6 |
| Total Solvents | Kg | 746 |
| Total Spray Solution Weight | Kg | 833 |

The mixture temperature was adjusted to a range of 20-45° C. and mixed until it was substantially homogenous and all components were substantially dissolved.

A spray drier, Niro PSD4 Commercial Spray Dryer, fitted with pressure nozzle (Spray Systems Maximum Passage series SK-MFP having orifice/core size 54/21) equipped with anti-bearding cap, was used under normal spray drying mode, following the dry spray process parameters recited in the table below.

### Processing parameters to generate spray dried dispersion of amorphous Compound III

| Parameter | Value | |
|---|---|---|
| Feed Pressure | 20 | bar |
| Feed Flow Rate | 92-100 | Kg/hr |
| Inlet Temperature | 93-99° | C. |
| Outlet Temperature | 53-57° | C. |
| Vacuum Dryer Temperature | 80° C. for 2 hours then | |
| | 110° C. (+/−5° C.) | |
| Vacuum Drying Time | 20-24 | hours |

A high efficiency cyclone separated the wet product from the spray gas and solvent vapors. The wet product contained 8.5-9171 MEK and 0.56-0.830 water and had a mean particle size of 17 −19 μm and a bulk density of 0.27-0.33 g/cc. The wet product was transferred to a 4000 L stainless steel double cone vacuum dryer for drying to reduce residual solvents to a level of less than about 5000 ppm and to generate dry Intermediate 1. The dry dispersion contained <0.030% MEK and 0.300 water.

### Example 4: Preparation of Solid Dispersion of Amorphous Compound II and Amorphous Compound III (1:1) Without Polymer

The production of solid dispersions of comprising both Compound II and Compound III is described in International Patent Publication WO 2015/160787, hereby incorporated by reference in its entirety.

56.5 g of Compound II and 56.5 g Compound III were added to 895.9 g of 90:10 methyl ethyl ketone (MVEK): water in a 2 L amber bottle. The material was stirred until both compounds had dissolved and was spray dried using a Buchi Mini Spray Dryer set to the parameters described in the table below.

### Compound II/Compound III spray dried dispersion processing parameters

| | |
|---|---|
| Formulation Description: | Compound II/Compound III (50/50) |

| T inlet (setpoint) | 115° | C. |
|---|---|---|
| T outlet (start) | 56° | C. |
| T outlet (end) | 42° | C. |
| Nitrogen Pressure | 120 | psi |
| Aspirator | 100% | |
| Pump | 40% | |
| Rotometer | 40 | mm |
| Filter Pressure | −60 | mbar |
| Condenser Temp | −20° | C. |

| | |
|---|---|
| Run Time | 1 h 17 min |

The material was secondary dried in a vacuum oven with nitrogen purge for 2 hours at 60° C. and overnight at 80° C.

The following are exemplary formulations of Compound I, Compound II, and Compound III.

### Example 5: Exemplary Formulation 1

Formulation 1

| | | | Content |
|---|---|---|---|
| | Component | Content | (mg/unit |
| Component | Function | (% w/w) | dose) |
| Crystalline Compound I | Active Ingredient | 15.61 | 100.00 |
| Form A | | | |
| Compound II SDD | Active Ingredient | 9.75 | 62.46 |
| (800 mg/g)^{a} | | | |
| Compound III SDD | Active Ingredient | 14.63 | 93.72 |
| (800 mg/g) ^{b} | | | |
| Microcrystalline Cellulose | Diluent/Binder | 50.07 | 320.75 |
| Sucralose Powder | Flavor | 3.94 | 25.24 |
| Croscarmellose Sodium | Disintegrant | 5.00 | 32.03 |
| Colloidal Silicon Dioxide | Flow Aid | 1.00 | 6.41 |
| Total | | 100.0 | 640.61 |

^{a}Compound II SDD Composition: 80%/20% wt tezacaftor/HPMC
^{b} Compound III SDD Composition: 80%/19.5%/0.5% wt ivacaftor/HPMCAS/SLS

The target weight of each granule was 7.0 mg with an average target weight of ten granules at 70.0 mg.

### Example 6: Exemplary Formulation 2

The target for each granule was approximately 2 mm in diameter and 7 mg in weight. With a target tablet weight of 7 mg, a total unit dose consists of 183 granules.

Formulation 2

| | | | Content |
|---|---|---|---|
| | Component | Content | (mg/unit |
| Component | Function | (% w/w) | dose) |
| Crystalline Compound I | Active Ingredient | 7.806 | 100.00 |
| Form A | | | |
| Compound II SDD | Active Ingredient | 4.879 | 62.50 |
| (800 mg/g) ^{c} | | | |
| Compound III SDD | Active Ingredient | 7.319 | 93.75 |
| (800 mg/g) ^{d} | | | |
| Mannitol | Diluent/Binder | 17.948 | 229.91 |
| Lactose Monohydrate | Diluent/Binder | 53.845 | 689.75 |
| Sucralose Powder | Flavor | 0.703 | 9.01 |
| Croscarmellose Sodium | Disintegrant | 5.000 | 64.05 |
| Colloidal Silicon Dioxide | Flow Aid | 1.000 | 12.81 |
| Magnesium Stearate | Lubricant | 1.500 | 19.22 |
| Total | | 100.0 | 1281.00 |

^{c} Compound II SDD Composition: 80%/20% wt tezacaftor/HPMC
^{d} Compound III Composition: 80%/19.5%/0.5% wt ivacaftor/HPMCAS/SLS

### Example 7: Exemplary Formulation 3

The target for each granule was 2 mm in diameter and 7 mg in weight. With a target tablet weight of 7 mg, a total unit dose consists of about 90 granules.

Formulation 3

| | | | Content |
|---|---|---|---|
| | Component | Content | (mg/unit |
| Component | Function | (% w/w) | dose) |
| Crystalline Compound I | Active Ingredient | 15.612 | 100.00 |
| Form A | | | |
| Compound II SDD | Active Ingredient | 9.758 | 62.50 |
| (800 mg/g) ^{e} | | | |
| Compound III SDD | Active Ingredient | 14.638 | 93.75 |
| (800 mg/g) ^{f} | | | |
| Mannitol | Diluent/Binder | 12.772 | 81.80 |
| Lactose Monohydrate | Diluent/Binder | 38.315 | 245.40 |
| Sucralose Powder | Flavor | 1.405 | 9.00 |
| Croscarmellose Sodium | Disintegrant | 5.000 | 32.036 |
| Colloidal Silicon Dioxide | Flow Aid | 1.000 | 6.41 |
| Magnesium Stearate | Lubricant | 1.500 | 9.61 |
| Total | | 100.0 | 640.5 |

^{e} Compound II SDD Composition: 80%/20% wt tezacaftor/HPMC
^{f} Compound III SDD Composition: 80%/19.5%/0.5% wt ivacaftor/HPMCAS/SLS

### Example 8: Exemplary Formulation 4

Formulation 4

| | | | Content |
|---|---|---|---|
| | Component | Content | (mg/unit |
| Component | Function | (% w/w) | dose) |
| Crystalline Compound I | Active Ingredient | 15.873 | 100.00 |
| Form A | | | |
| Compound II SDD | Active Ingredient | 9.921 | 62.50 |
| (800 mg/g) ^{g} | | | |
| Compound III SDD | Active Ingredient | 14.881 | 93.75 |
| (800 mg/g) ^{h} | | | |
| Mannitol | Diluent/Binder | 12.599 | 79.38 |
| Lactose Monohydrate | Diluent/Binder | 37.797 | 238.12 |
| Sucralose Powder | Flavor | 1.429 | 9.00 |
| Croscarmellose Sodium | Disintegrant | 5.000 | 31.50 |
| Colloidal Silicon Dioxide | Flow Aid | 1.000 | 6.30 |
| Magnesium Stearate | Lubricant | 1.500 | 9.45 |
| Total | | 100.0 | 630.0 |

^{g} Compound II SDD Composition: 80%/20% wt tezacaftor/HPMC
^{h} Compound III SDD Composition: 80%/19.5%/0.5% wt ivacaftor/HPMCAS/SLS

### Example 9: Exemplary Formulation 5

Formulation 5

| | | | Content |
|---|---|---|---|
| | Component | Content | (mg/unit |
| Component | Function | (% w/w) | dose) |
| Crystalline Compound I | Active Ingredient | 15.873 | 100.00 |
| Form A | | | |
| Compound II SDD | Active Ingredient | 9.921 | 62.50 |
| (800 mg/g) ⁱ | | | |
| Compound III SDD | Active Ingredient | 14.881 | 93.75 |
| (800 mg/g) ^{j} | | | |
| Mannitol | Diluent/Binder | 12.474 | 78.59 |
| Lactose Monohydrate | Diluent/Binder | 37.422 | 235.76 |
| Sucralose Powder | Flavor | 1.429 | 9.00 |
| Croscarmellose Sodium | Disintegrant | 6.000 | 37.80 |
| Colloidal Silicon Dioxide | Flow Aid | 1.000 | 6.30 |
| Magnesium Stearate | Lubricant | 1.000 | 6.30 |
| Total | | 100.0 | 630.0 |

ⁱ Compound II SDD Composition: 80%/20% wt tezacaftor/HPMC
^{j} Compound III SDD Composition: 80%/19.5%/0.5% wt ivacaftor/HPMCAS/SLS

What is claimed is:

1. A pharmaceutical composition comprising:
a. Compound I;
b. Compound II;
c. Compound III;
d. one or more fillers;
e. a disintegrant;
f. a sweetener;
g. a glidant; and
h. a lubricant.

2. The pharmaceutical composition of claim 1, wherein Compound I is crystalline Compound I Form A.

3. The pharmaceutical composition of claim 1 or claim 2, wherein Compound II is amorphous or substantially amorphous Compound II.

4. The pharmaceutical composition of any one of claims 1 to 3, wherein Compound III is amorphous or substantially amorphous Compound II.

5. The pharmaceutical composition of any one of claims 1 to 4, wherein Compound II and Compound III are in a single solid dispersion without polymer.

6. The pharmaceutical composition of any one of claims 1 to 4, wherein Compound II and Compound III are in separate solid dispersions, each containing a polymer.

7. The pharmaceutical composition of claim 6, wherein the solid dispersion comprising Compound II further comprises HPMC.

8. The pharmaceutical composition of claim 6 or claim 7, wherein the solid dispersion comprising Compound III further comprises HPMCAS.

9. The pharmaceutical composition of claim 6, wherein the solid dispersion comprising Compound II comprises about 80 wt % of substantially amorphous or amorphous Compound II by weight of the dispersion and about 20 wt % of IPMC by weight of the dispersion.

10. The pharmaceutical composition of claim 6 or claim 9, wherein the solid dispersion comprising Compound III contains about 80 wt % of substantially amorphous or amorphous Compound III by weight of the dispersion, about 19.5 wt % of HPMCAS by weight of the dispersion, and about 0.5 wt % SLS by weight of the dispersion.

11. The pharmaceutical composition of any one of claims 1 to 10, wherein the one or more fillers comprises mannitol.

12. The pharmaceutical composition of claim 11, wherein the mannitol is present in an amount of about 10 to about 14 percent by weight of the composition.

13. The pharmaceutical composition of any one of claims 1 to 12, wherein the one or more fillers comprise lactose.

14. The pharmaceutical composition of claim 13, wherein lactose is present in an amount of about 35 to about 40 percent by weight of the composition.

15. The pharmaceutical composition of claim 13, wherein the filler is a binary filler composed of mannitol and lactose.

16. The pharmaceutical composition of any of claims 1 to 15, wherein the disintegrant is croscarmellose sodium.

17. The pharmaceutical composition of claim 16, wherein the croscarmellose sodium is present in an amount of about 6 percent by weight of the pharmaceutical composition.

18. The pharmaceutical composition of any one of claims 1 to 17, wherein the sweetener is sucralose.

19. The pharmaceutical composition of claim 18, where in the sucralose is present in an amount of about 1 to about 2 percent by weight of the pharmaceutical composition.

20. The pharmaceutical composition of any one of claims 1 to 19, wherein the lubricant is magnesium stearate.

21. The pharmaceutical composition of claim 20, wherein the magnesium stearate is present in an amount of about 0.5 to about 1.5 percent by weight of the composition.

22. The pharmaceutical composition of any one of claims 1 to 21, wherein the glidant is colloidal silicon dioxide.

23. The pharmaceutical composition of claim 22, wherein the colloidal silicon dioxide is present in an amount of about 0.5 to about 1.5 percent by weight of the composition.

24. A pharmaceutical composition comprising:
about 14 to about 17 wt % crystalline Compound I Form A by weight of the composition,
about 8 to about 11 wt % of a solid dispersion by weight of the composition, wherein the solid dispersion comprises 80% amorphous or substantially amorphous Compound II and 20% HPMC,
about 14 to about 16 wt % by weight of a solid dispersion by weight of the composition, wherein the solid dispersion comprises 80% amorphous or substantially amorphous Compound III and 19.5% HPMCAS and 0.5% SLS,
about 48 to about 52 wt % microcrystalline cellulose by weight of the composition,
about 3 to about 5 wt % of sucralose by weight of the composition,
about 4 to about 6 wt % croscarmellose sodium by weight of the composition, and
about 0.5 to about 1.5 wt % colloidal silicon dioxide by weight of the composition.

25. A pharmaceutical composition comprising:
about 6 to about 9 wt % crystalline Compound I Form A by weight of the composition,
about 4 to about 6 wt % of a solid dispersion by weight of the composition, wherein the solid dispersion comprises 80% amorphous or substantially amorphous Compound II and 20% HPMC,
about 6 to about 8 wt % by weight of a solid dispersion composed of 80% amorphous or substantially amorphous Compound III and 19.5% HPMCAS and 0.5% SLS,
about 16 to about 19 wt % mannitol by weight of the composition,
about 52 to about 55 wt % lactose by weight of the composition,
about 0.5 to about 1.0 wt % of sucralose by weight of the composition,
about 4 to about 6 wt % croscarmellose sodium by weight of the composition,
about 0.5 to about 1.5 wt % colloidal silicon dioxide by weight of the composition, and
about 1 to about 2 wt % magnesium stearate by weight of the composition.

26. A pharmaceutical composition comprising:
about 14 to about 17 wt % crystalline Compound I Form A by weight of the composition,
about 8 to about 11 wt % of a solid dispersion by weight of the composition, wherein the solid dispersion comprises 80% amorphous or substantially amorphous Compound II and 20% HPMC,
about 14 to about 16 wt % by of a solid dispersion by weight of the composition, wherein the solid dispersion comprises 80% amorphous or substantially amorphous Compound III and 19.5% HPMCAS and 0.5% SLS,
about 11 to about 14 wt % mannitol by weight of the composition,
about 36 to about 40 wt % lactose by weight of the composition,
about 1.0 to about 2.0 wt % of sucralose by weight of the composition,
about 4 to about 6 wt % croscarmellose sodium by weight of the composition,
about 0.5 to about 1.5 wt % colloidal silicon dioxide by weight of the composition, and
about 1 to about 2 wt % magnesium stearate by weight of the composition.

27. A pharmaceutical composition comprising:
about 14 to about 17 wt % crystalline Compound I Form A by weight of the composition,
about 8 to about 11 wt % of a solid dispersion by weight of the composition, wherein the solid dispersion comprises 80% amorphous or substantially amorphous Compound II and 20% HPMC,
about 14 to about 16 wt % by of a solid dispersion by weight of the composition, wherein the solid dispersion comprises 80% amorphous or substantially amorphous Compound III and 19.5% HPMCAS and 0.5% SLS,
about 11 to about 14 wt % mannitol by weight of the composition,
about 37 to about 40 wt % lactose by weight of the composition,
about 1.0 to about 2.0 wt % of sucralose by weight of the composition,
about 4 to about 6 wt % croscarmellose sodium by weight of the composition,
about 0.5 to about 1.5 wt % colloidal silicon dioxide by weight of the composition, and
about 1 to about 2 wt % magnesium stearate by weight of the composition.

28. A pharmaceutical composition comprising:
about 14 to about 17 wt % crystalline Compound I Form A by weight of the composition,
about 8 to about 11 wt % of a solid dispersion composed of 80% amorphous or substantially amorphous Compound II and 20% HPMC,
about 14 to about 16 wt % by weight of a solid dispersion by weight of the composition, wherein the solid dispersion comprises 80% amorphous or substantially amorphous Compound III and 19.5% HPMCAS and 0.5% SLS,
about 11 to about 14 wt % mannitol by weight of the composition,
about 36 to about 40 wt % lactose by weight of the composition,
about 1.0 to about 2.0 wt % of sucralose by weight of the composition,
about 4 to about 8 wt % croscarmellose sodium by weight of the composition,
about 0.5 to about 1.5 wt % colloidal silicon dioxide by weight of the composition, and
about 0.5 to about 1.5 wt % magnesium stearate by weight of the composition.

29. The pharmaceutical composition of any of claims 1 to 28, wherein the pharmaceutical composition is a unit dose form comprising a plurality of granules, pellets, particles or mini-tablets, and wherein the unit dose form comprises from about 20 mg to about 100 mg of crystalline Compound I Form A.

30. The pharmaceutical composition of any of claims 1 to 29, wherein the pharmaceutical composition is a unit dose form comprising a plurality of granules, pellets, particles or mini-tablets, and wherein the unit dose form comprises from about 10 mg to about 50 mg of amorphous or substantially amorphous Compound II.

31. The pharmaceutical composition of any of claims 1 to 30, wherein the pharmaceutical composition is a unit dose form comprising a plurality of granules, pellets, particles or mini-tablets, and wherein the unit dose form comprises from about 15 mg to about 75 mg of amorphous or substantially amorphous Compound III.

32. The pharmaceutical composition of any of claims 1 to 31, wherein the pharmaceutical composition is a unit dose form comprising a plurality of granules, pellets, particles or mini-tablets, and wherein the unit dose form comprises about 100 mg of crystalline Compound I Form A, about 50 mg of amorphous or substantially amorphous Compound II, and about 75 mg of amorphous or substantially amorphous Compound III.

33. The pharmaceutical composition of any of claims 1 to 31, wherein the pharmaceutical composition is a unit dose form comprising a plurality of granules, pellets, particles or mini-tablets, and wherein the unit dose form comprises about 80 mg of crystalline Compound I Form A, about 40 mg of amorphous or substantially amorphous Compound II, and about 60 mg of amorphous or substantially amorphous Compound III.

34. The pharmaceutical composition of any of claims 1 to 31, wherein the pharmaceutical composition is a unit dose form comprising a plurality of granules, pellets, particles or mini-tablets, and wherein the unit dose form comprises about 20 mg of crystalline Compound I Form A, about 10 mg of amorphous or substantially amorphous Compound II, and about 15 mg of amorphous or substantially amorphous Compound III.

35. The pharmaceutical composition of any of claims 1 to 34, wherein the unit dose form comprises from about 18 to about 90 mini-tablets.

36. The pharmaceutical composition of any of claims 1 to 34, wherein the unit dose form comprises about 90 mini-tablets.

37. The pharmaceutical composition of any of claims 1 to 34, wherein the unit dose form comprises about 72 mini-tablets.

38. The pharmaceutical composition of any of claims 1 to 34, wherein the unit dose form comprises about 18 mini-tablets.

39. The pharmaceutical composition of any of claims 1 to 38, wherein the composition is in the form of a granule or a mini-tablet with a shape that is cylinder-like, oval-like, cone-like, sphere-like, ellipsis-like, polygon-like or combinations thereof, wherein the granule or mini-tablet has as its longest dimension or diameter a length of about 2 mm.

40. A method of treating or lessening the severity of CFTR-mediated disease in a pediatric patient comprising administering to the pediatric patient a pharmaceutical composition of any of claims 1-39.

41. The method of claim 40, wherein the CFTR mediated disease is cystic fibrosis

42. The method of claim 40 or claim 41, wherein the patient weighs about 14 or more kilograms.

43. The method claim 40 or claim 41, wherein the patient weighs less than 14 kilograms.
